(19) United States
(12) Patent Application Publication
(10) Pub. No.: US 2024/0301475 A1
(43) Pub. Date: Sep. 12, 2024

(54) METHODS AND COMPOSITIONS FOR DETECTION USING NUCLEIC ACID PROBES

(71) Applicant: 10x Genomics, Inc., Pleasanton, CA (US)

(72) Inventors: Paulius MIELINIS, Hagersten (SE); Jessica ÖSTLIN, Stocksund (SE)

(21) Appl. No.: 18/431,793

(22) Filed: Feb. 2, 2024

Related U.S. Application Data
(60) Provisional application No. 63/443,279, filed on Feb. 3, 2023.

(51) Int. Cl. C12Q 1/682

(52) U.S. Cl. CPC C12Q 1/682

(57) ABSTRACT

The present disclosure relates in some aspects to methods and compositions for detecting analytes in situ in a biological sample via a nucleic acid hybridization complex comprising probes with spacer regions.

### CROSS-REFERENCE TO RELATED APPLICATIONS

This application claims priority to U.S. provisional Patent Application No. 63/443,279, filed Feb. 3, 2023, entitled “METHODS AND COMPOSITIONS FOR DETECTION USING NUCLEIC ACID PROBES”, which is herein incorporated by reference in its entirety for all purposes.

### FIELD

The present disclosure relates in some aspects to methods and compositions for in situ analysis of an analyte in a biological sample.

### BACKGROUND

Genomic, transcriptomic, and proteomic profiling of cells and tissue samples using microscopic imaging can resolve multiple analytes of interest at the same time, thereby providing valuable information regarding analyte abundance and localization in situ. Thus, in situ assays are important tools, for example, for understanding the molecular basis of cell identity and developing treatment for diseases. In some aspects, existing methods are limited by the ability to provide sufficient signal amplification for efficient detection. Improved probe designs and methods are therefore needed. Provided herein are methods and compositions that address such and other needs

### SUMMARY

In some aspects, provided herein is a method for analyzing a biological sample, comprising: (a) contacting the biological sample with: (i) an amplifier probe comprising a recognition sequence and at least two reporter hybridization regions, wherein the at least two reporter hybridization regions are separated by a spacer region of between 4 and 8 nucleotides in length, and (ii) a plurality of reporter probes, wherein each reporter probe of at least a subset of the reporter probes comprises a detectable label and a reporter region capable of hybridizing to the reporter hybridization region; and (b) detecting a signal from a complex formed by hybridization of at least two reporter probes to the at least two reporter hybridization regions.

In some aspects, provided herein is a kit, comprising: (i) an amplifier probe comprising a recognition sequence and at least two reporter hybridization regions, wherein the at least two reporter hybridization regions are separated by a spacer region of between 4 and 8 nucleotides in length, and (ii) a plurality of reporter probes, wherein each reporter probe of at least a subset of the reporter probes comprises a detectable label and a reporter region capable of hybridizing to the reporter hybridization region.

In some of any of the embodiments herein, each reporter hybridization region is separated from the other reporter hybridization regions by a spacer region of between 4 and 8 nucleotides in length, wherein the spacer regions are the same or different sequences.

In any of the embodiments herein, the amplifier probe can comprise, from 5′ to 3′ or from 3′ to 5′, a first reporter hybridization region, a first spacer region, and a second reporter hybridization region. In any of the embodiments herein, the amplifier probe can comprise, from 5′ to 3′ or from 3′ to 5′, a first reporter hybridization region, a first spacer region, a second reporter hybridization region, a second spacer region, and a third reporter hybridization region. In any of the embodiments herein, the amplifier probe can comprise, from 5′ to 3′ or from 3′ to 5′, the recognition sequence, a first reporter hybridization region, a first spacer region, and a second reporter hybridization region. In any of the embodiments herein, the amplifier probe can comprise, from 5′ to 3′ or from 3′ to 5′, the recognition sequence, a first reporter hybridization region, a first spacer region, a second reporter hybridization region, a second spacer region, and a third reporter hybridization region. In any of the embodiments herein, the amplifier probe can further comprise a linker sequence of between 2 and 8 nucleotides in length between the recognition sequence and the first reporter hybridization region.

In any of the embodiments herein, the spacer region is a random sequence of nucleotides. In any of the embodiments herein, the spacer region can be a sequence of thymidines and/or adenines. In some embodiments, the spacer region is a sequence of only thymidines. In some embodiments, the spacer region is a sequence of only adenines. In some embodiments, the spacer region is a sequence of only thymidines and adenines. In some embodiments, the spacer region is a sequence of thymidines. In any of the embodiments herein, each spacer region can be a sequence of thymidines and/or adenines, and wherein the spacer regions are the same or different.

In some aspects, provided herein is a method for analyzing a biological sample, comprising: (a) contacting the biological sample with: (i) an amplifier probe comprising a recognition sequence and at least two reporter hybridization regions, wherein the at least two reporter hybridization regions are separated by a spacer region comprising one or more organic non-nucleic acid spacers, and (ii) a plurality of reporter probes, wherein each reporter probe of at least a subset of the reporter probes comprises a detectable label and a reporter region capable of hybridizing to the reporter hybridization region; and (b) detecting a signal from a complex formed by hybridization of at least two reporter probes to the at least two reporter hybridization regions. In some embodiments, the spacer region comprises one organic non-nucleic acid spacer. In some embodiments, the spacer region comprises two or more organic non-nucleic acid spacers.

In some aspects, provided herein is a kit, comprising: (i) an amplifier probe comprising a recognition sequence and at least two reporter hybridization regions, wherein the at least two reporter hybridization regions are separated by a spacer region comprising one or more organic spacers, and (ii) a plurality of reporter probes, wherein each reporter probe of at least a subset of the reporter probes comprises a detectable label and a reporter region capable of hybridizing to the reporter hybridization region. In some embodiments, the kit further comprises a labeling agent conjugated to a oligonucleotide reporter, wherein the recognition sequence of the amplifier probe binds to a sequence of the oligonucleotide reporter. In some embodiments, the labeling agent comprises an antibody or epitope binding fragment thereof. In some embodiments, the kit comprise a plurality of labeling agents each conjugated to an oligonucleotide reporter.

In any of the embodiments herein, the one or more organic non-nucleic acid spacers can be selected from the group consisting of a triethylene glycol spacer, a C3 spacer phosphoramidite, a hexanediol, an 18-atom hexa-ethyleneglycol spacer, a glycol linker, a carbon chain, a polyethylene glycol (PEG) spacer, and combinations thereof. In any of the embodiments herein, the one or more organic non-nucleic acid spacers can comprise an 18-atom hexa-ethyleneglycol spacer. In any of the embodiments herein, the amplifier probe can comprise, from 5′ to 3′ or from 3′ to 5′, a first reporter hybridization region, a first spacer region, a second reporter hybridization region, a second spacer region, and a third reporter hybridization region. In any of the embodiments herein, the amplifier probe can comprise, from 5′ to 3′ or from 3′ to 5′, the recognition sequence, a first reporter hybridization region, a first spacer region, and a second reporter hybridization region. In any of the embodiments herein, the amplifier probe can comprise, from 5′ to 3′ or from 3′ to 5′, the recognition sequence, a first reporter hybridization region, a first spacer region, a second reporter hybridization region, a second spacer region, and a third reporter hybridization region. In any of the embodiments herein, the spacer region may no more than about 10 nm in length. In any of the embodiments herein, the spacer region may be at least 2.5, 3, 4, or 5 nm in length.

In some of any of the embodiments herein, the amplifier probe comprises at least 3, 4, 5 or more reporter hybridization regions. In any of the embodiments herein, the amplifier probe can comprise between 2 and 20 reporter hybridization regions. In any of the embodiments herein, the reporter hybridization regions can be copies of the same nucleic acid sequence. In any of the embodiments herein, the reporter hybridization regions can be individually between 15 and 25 nucleotides in length. In any of the embodiments herein, the reporter probes can be individually between 15 and 25 nucleotides in length. In any of the embodiments herein, the reporter region can be between 15 and 25 nucleotides in length.

In some of any of the embodiments herein, the detectable label is a fluorophore. In any of the embodiments herein, the fluorophore may have an excitation peak between 480 nm and 500 nm. In any of the embodiments herein, the fluorophore may have an excitation peak between 520 nm and 540 nm. In any of the embodiments herein, the fluorophore can have an excitation peak between 590 nm and 600 nm. In any of the embodiments herein, the fluorophore may have an excitation peak between 640 nm and 660 nm.

In any of the embodiments herein, the detectable label can be linked to the reporter region by a disulfide. In any of the embodiments herein, the detectable label can be linked to the reporter region by one or more nucleotides.

In any of the embodiments herein, the signal from the detectable label of the reporter probe detected in (b) may be brighter than a control signal of a reporter probe having the same detectable label hybridized to a control amplifier probe that does not comprise the spacer regions. In any of the embodiments herein, the signal from the detectable label of the reporter probe detected in (b) may be brighter than a control signal of a reporter probe having the same detectable label hybridized to a control amplifier probe that comprises a dinucleotide spacer region between each of at least two reporter hybridization regions in the control amplifier probe. In any of the embodiments herein, the signal-to-noise ratio from the detectable label of the reporter probe detected in (b) may be brighter than a control signal-to-noise ratio for a reporter probe having the same detectable label hybridized to a control amplifier probe that does not comprise the spacer regions. In any of the embodiments herein, wherein the signal-to-noise ratio from the detectable label of the reporter probe detected in (b) may be greater than a control the signal-to-noise ratio for a reporter probe having the same detectable label hybridized to a control amplifier probe that comprises a dinucleotide spacer region between each of at least two reporter hybridization regions in the control amplifier probe. In any of the embodiments herein, the signal-to-noise ratio from the detectable label of the reporter probe detected in (b) may be at least 10%, at least 20%, at least 30%, at least 40%, at least 50%, or at least 55% higher than the signal-to-noise ratio for the reporter probe having the same detectable label hybridized to the control amplifier probe.

In any of the embodiments herein, the recognition sequence may be complementary to an amplifier probe hybridization region in an adapter probe. In some embodiments, the method comprises contacting the biological sample with the adapter probe. In any of the embodiments herein, the adapter probe can comprise multiple copies of the amplifier probe hybridization region. In any of the embodiments herein, multiple copies of the amplifier probe may be hybridized to the adapter probe. In any of the embodiments herein, the complex can comprises between 2 and 20 amplifier probes hybridized to the adapter probe in the biological sample.

In some of any of the embodiments herein, the multiple copies of the amplifier probe hybridization region are separated by a linker. In any of the embodiments herein, the linker can be a nucleotide sequence of between 2 and 10 nucleotides in length. In any of the embodiments herein, the linker can be a sequence of between 4 and 8 nucleotides in length. In any of the embodiments herein, the linker can be a random sequence of nucleotides. In any of the embodiments herein, the linker can be a sequence of thymidines and/or adenines. In any of the embodiments herein, the linker can be a sequence of thymidines.

In some of any of the embodiments herein, the signal detected from the complex (e.g., in (b)) is a signal from the detectable labels of at least 10, at least 20, at least 30, or at least 40 reporter probes bound in the complex. In some embodiments, the signal detected from the complex is a signal from the detectable labels of at least 3, at least 4, at least 5, or at least 6 reporter probes bound in the complex. In some embodiments, the signal detected from the complex is a signal from the detectable labels of at least 2 reporter probes bound in the complex. In some embodiments, the signal detected from the complex is a signal from the detectable labels of at least 3 reporter probes bound in the complex.

In any of the embodiments herein, the adapter probe can be between about 50 and about 500 nucleotides in length. In any of the embodiments herein, the amplifier probe can be between about 50 and about 500 nucleotides in length. In some embodiments, the amplifier probe is between about 20 and about 200 nucleotides in length. In any of the embodiments herein, the adapter probe can comprise an adapter sequence capable of binding directly or indirectly to a primary probe hybridized to a target nucleic acid in the biological sample. In some embodiments, the method comprises contacting the biological sample with the primary probe.

In any of the embodiments herein, the recognition sequence can be complementary to an amplifier probe hybridization region in a primary probe hybridized to a target nucleic acid in the biological sample. In some embodiments, the method comprises contacting the biological sample with the primary probe. In any of the embodiments herein, the primary probe can be between about 50 and about 500 nucleotides in length. In any of the embodiments herein, the primary probe can comprise a targeting sequence complementary to a target sequence in a target nucleic acid in the biological sample. In any of the embodiments herein, the recognition sequence can be complementary to a target sequence in a target nucleic acid in the biological sample.

In any of the embodiments herein, the detectable label can be a first detectable label, and the reporter probe can further comprise a second detectable label. In some embodiments, the first detectable label and the second detectable label are different. In some embodiments, (b) comprises detecting a first signal from the first detectable label and detecting a second signal from the second detectable label. In any of the embodiments herein, the first detectable label can be at the 5′ end of the reporter probe and the second detectable label is at the 3′ end of the reporter probe. In any of the embodiments herein, the reporter probe can comprise a flexible linker between the reporter region and the detectable label (e.g., the first detectable label). In any of the embodiments herein, the reporter probe can comprise a flexible linker between the reporter region and the second detectable label.

In any of the embodiments herein, the target nucleic acid can be a cellular nucleic acid analyte or a product thereof. In any of the embodiments herein, the target nucleic acid can be associated with a non-nucleic acid analyte. In some embodiments, the target nucleic acid is an oligonucleotide reporter in a labeling agent that binds to the analyte. In any of the embodiments herein, the labeling agent can be an antibody conjugated to the oligonucleotide reporter. In some embodiments, the non-nucleic acid analyte is a protein. In any of the embodiments herein, the target nucleic acid can be RNA. In some embodiments, the target nucleic acid is mRNA. In some embodiments, the target nucleic acid is an RNA fragment. In any of the embodiments herein, the target nucleic acid can be a probe or probe set associated with a nucleic acid analyte or product thereof in the biological sample.

In any of the embodiments herein, the target nucleic acid can be a rolling circle amplification product. In some embodiments, the target nucleic acid is a rolling circle amplification product generated at a location in the biological sample. In any of the embodiments herein, the method can comprise generating the rolling circle amplification product from a circular or circularized probe or probe set associated with an analyte in the biological sample. In any of the embodiments herein, the analyte can be a nucleic acid analyte and the circular or circularized probe or probe set can be associated with the analyte by binding directly or indirectly to the nucleic acid analyte. In any of the embodiments herein, the analyte can be a non-nucleic acid analyte and the circular or circularized probe or probe set can be associated with the analyte by binding directly or indirectly to an oligonucleotide reporter in a labeling agent that binds to the analyte.

In any of the embodiments herein, the biological sample may be non-homogenized and optionally selected from the group consisting of a formalin-fixed, paraffin-embedded (FFPE) sample, a frozen tissue sample, and a fresh tissue sample. In any of the embodiments herein, the biological sample can be permeabilized. In any of the embodiments herein, the biological sample can be embedded in a matrix. In some embodiments, the matrix comprises a hydrogel. In any of the embodiments herein, the biological sample can be cleared. In any of the embodiments herein, the biological sample can be a tissue slice between about 1 μm and about 50 μm in thickness. In any of the embodiments herein, the tissue slice can be between about 5 μm and about 35 μm in thickness. In any of the embodiments herein, the complex can be detected at a location in the biological sample. In any of the embodiments herein, the biological sample can be on a substrate.

### BRIEF DESCRIPTION OF THE DRAWINGS

The drawings illustrate certain features and advantages of this disclosure. These embodiments are not intended to limit the scope of the appended claims in any manner.

FIG. 1 depicts an exemplary branched probe design, comprising: a primary probe containing a targeting sequence and an adapter probe hybridization region separated by a linker sequence (L); an adapter probe containing an adapter region and a plurality of amplifier probe binding regions, all separated by a linker sequence (L); an amplifier probe containing a recognition sequence and a plurality of reporter hybridization regions each separated by a spacer region (S); and a plurality of reporter probes containing a reporter region and a detectable label. Asterisks indicate additional hybridized amplifier probes bound to the adapter probe (omitted for simplicity).

FIG. 2 provides in situ fluorescence microscopy images of biological samples in which the expression of Proxy1 mRNA was detected using a branched probe design where the spacer sequences within the adapter probes and amplifier probes were modified to contain a sequence of either 2 thymidine nucleotides, the 18-atom hexa-ethyleneglycol spacer iSp18, or a sequence of 5 thymidine nucleotides. Proxy1 mRNA expression was detected by red fluorophore conjugated detection oligos.

FIG. 3 provides quantification of fluorescence intensity measurements from FIG. 2. Proxy1 mRNA expression was detected by red fluorophore conjugated detection oligos.

FIG. 4 provides in situ fluorescence microscopy images of mouse brain tissue sections in which the expression of Proxy1 mRNA was detected using a branched probe design comprising spacer region sequences in the amplifier and adapter probes of 2 thymidine nucleotides, 5 thymidine nucleotides, or 8 thymidine nucleotides. Proxy1 mRNA expression was detected by red fluorophore conjugated detection oligos.

FIG. 5 provides in situ fluorescence microscopy images of mouse brain tissue sections in which the expression of Proxy1 mRNA was detected using a branched probe design comprising spacer regions of 2 thymidine nucleotides or 5 thymidine nucleotides where the detection oligonucleotide conjugated to green, yellow, orange or red fluorophores.

FIG. 6 provides quantification of fluorescence intensity measurements from FIG. 5. Proxy1 mRNA expression was detected by detection oligos conjugated to green, yellow, orange or red fluorophores.

FIG. 7 provides in situ fluorescence microscopy images of mouse brain tissue sections in which the expression of Proxy1 mRNA was detected using a branched probe design comprising adapter probes and amplifier probes containing spacer regions with a length of 2 thymidine nucleotides, 5 thymidine nucleotides, or 8 thymidine nucleotides.

FIG. 8 provides in situ fluorescence microscopy images of mouse brain tissue sections in which the expression of Proxy1 mRNA was detected using a branched probe design comprising primary probes containing spacer regions with a length of either 2 thymidine nucleotides or 5 thymidine nucleotides. The spacer length in the adapter and amplifier probes was either 2 thymidine nucleotides, 5 thymidine nucleotides, or 8 thymidine nucleotides.

### DETAILED DESCRIPTION

All publications, comprising patent documents, scientific articles and databases, referred to in this application are incorporated by reference in their entirety for all purposes to the same extent as if each individual publication were individually incorporated by reference. If a definition set forth herein is contrary to or otherwise inconsistent with a definition set forth in the patents, applications, published applications and other publications that are herein incorporated by reference, the definition set forth herein prevails over the definition that is incorporated herein by reference.

The section headings used herein are for organizational purposes only and are not to be construed as limiting the subject matter described.

### I. Overview

The present application provides probe designs and methods for formation of amplifier probe and reporter probe complexes with improved properties for in situ detection of target nucleic acid molecules and/or target analytes in a biological sample. In some embodiments, provided herein are amplifier probes comprising spacer regions of at least 4 nucleotides in length between reporter hybridization regions. Reporter probes individually comprise a reporter region complementary to the reporter hybridization region and a detectable label. When the reporter probes hybridize to the reporter hybridization regions in the amplifier probe, the spacer regions provide spacing between the hybridized reporter probes.

The present application further provides data demonstrating increased signal intensity and/or signal-to-noise ratios for amplifier probe and reporter probe complexes with spacer regions longer than 4 nucleotides in length (e.g., 5T or 8T spacers) between the reporter hybridization regions. Without being bound by theory, in some aspects the increased spacing between the detectable labels of reporter probes (e.g., between fluorescent dyes of the reporter probes) reduces or avoids auto-quenching of the detectable labels. In some embodiments, spacing out two reporter probes from each other avoids steric effects that reduce the efficiency of reporter probe hybridization. For example, without being bound by theory, reporter probes that are too close might not sit well close to each other, especially give one probe has a detectable label (e.g., a 5′-dye) attached to it that might interfere with hybridization of another reporter probe's 3′ end.

In some embodiments, the detectable label is a fluorophore having an excitation peak between 480 nm and 500 nm. In some embodiments, the detectable label is a fluorophore having an excitation peak between 520 nm and 540 nm. In some embodiments, the detectable label is a fluorophore having an excitation peak between 590 nm and 600 nm. In some embodiments, the detectable label is a fluorophore having an excitation peak between 640 nm and 660 nm.

### II. Methods and Probes

In some aspects, provided herein is a method for analyzing a biological sample, comprising: (a) contacting the biological sample with: (i) an amplifier probe comprising a recognition sequence and at least two reporter hybridization regions, wherein the at least two reporter hybridization regions are separated by a spacer region of between 4 and 8 nucleotides in length, and (ii) a plurality of reporter probes, wherein each reporter probe of at least a subset of the reporter probes comprises a detectable label and a reporter region capable of hybridizing to the reporter hybridization region; and (b) detecting a signal from a complex formed by hybridization of at least two reporter probes to the at least two reporter hybridization regions.

In some embodiments, provided herein is a method for analyzing a biological sample, comprising: contacting the biological sample with: (i) an amplifier probe comprising a recognition sequence and at least two reporter hybridization regions, wherein the at least two reporter hybridization regions are separated by a spacer region of between 4 and 8 nucleotides in length, (ii) a plurality of reporter probes, wherein each reporter probe of at least a subset of the reporter probes comprises a detectable label and a reporter region capable of hybridizing to the reporter hybridization region, and (iii) an adapter probe comprising at least two amplifier probe hybridization regions and an adapter region, wherein the adapter region is complementary to an adapter hybridization region in a primary probe or target nucleic acid in the biological sample, and the amplifier probe hybridization regions are complementary to the recognition sequence. In some embodiments, the method comprises contacting the biological sample with a primary probe comprising an adapter probe hybridization region that binds directly or indirectly to the adapter probe. In some embodiments, the method comprises detecting a signal from a complex formed by hybridization of the reporter probes, the amplifier probe(s), the adapter probe(s), and the primary probe or target nucleic acid. In some embodiments, the spacer regions in the amplifier probe(s) improve the signal intensity and/or signal-to-noise ratio (SNR) of the detected complex. In some embodiments, the detected signal intensity or SNR is greater compared to that detected for a control complex wherein the amplifier probes do not comprise spacer regions between the reporter probe hybridizations. In some embodiments, the detected signal intensity or SNR is greater compared to that detected for a control complex wherein the amplifier probes comprise spacer regions of 2 nucleotides in length or shorter between the reporter probe hybridizations. In some embodiments, the detected signal intensity or SNR is greater compared to that detected for a control complex wherein the amplifier probes comprise spacer regions of 3 nucleotides in length or shorter between the reporter probe hybridizations.

In some embodiments, the detectable label is a fluorophore having an excitation peak at about 488 nm, and the SNR of the detected complex is at least 5%, at least 10%, or at least 11% higher than the SNR for a control complex wherein the amplifier probes comprise spacer regions of 2 nucleotides in length or shorter between the reporter probe hybridizations. In some embodiments, the detectable label is a fluorophore having an excitation peak at about 532 nm, and the SNR of the detected complex is at least 40%, at least 45%, or at least 49% higher than the SNR for a control complex wherein the amplifier probes comprise spacer regions of 2 nucleotides in length or shorter between the reporter probe hybridizations. In some embodiments, the detectable label is a fluorophore having an excitation peak at about 590 nm, and the SNR of the detected complex is at least 40%, at least 45%, or at least 46% higher than the SNR for a control complex wherein the amplifier probes comprise spacer regions of 2 nucleotides in length or shorter between the reporter probe hybridizations. In some embodiments, the detectable label is a fluorophore having an excitation peak at about 647 nm, and the SNR of the detected complex is at least 40%, at least 50%, at least 55%, or at least 59% higher than the SNR for a control complex wherein the amplifier probes comprise spacer regions of 2 nucleotides in length or shorter between the reporter probe hybridizations.

In some embodiments, the method comprises contacting the biological sample with an amplifier probe comprising multiple copies of a reporter hybridization region separated by spacer regions, reporter probe molecules comprising a reporter region complementary to the reporter hybridization region, and a detectable label (such as a fluorophore), an adapter probe comprising multiple copies of an amplifier probe hybridization region separated by a linker region, and a primary probe comprising multiple copies of an adapter hybridization region separated by a linker region. In some embodiments, the spacer regions in the amplifier probes are between 5 nucleotides in length and 8 nucleotides in length. In some embodiments, the linker regions in the adapter probes are between 5 nucleotides in length and 8 nucleotides in length. In some embodiments, the linker regions in the primary probes are between 5 nucleotides in length and 8 nucleotides in length. In some embodiments, the spacer regions in the amplifier probes are each 5 nucleotides in length and the linker regions in the adapter probes and primary probes are each 5 nucleotides in length. In some embodiments, the spacer regions in the amplifier probes are each a sequence of five thymidines (e.g., 5T; TTTTT), and the linker regions in the adapter probes and primary probes are each a sequence of five thymidines (5T; TTTTT). In some embodiments, the linker regions in the primary probes are each 2 nucleotides in length, the spacer regions in the amplifier probes are each 8 nucleotides in length, and the spacer regions in the adapter probes are each 8 nucleotides in length. In some embodiments, the linker regions in the primary probes are each a sequence of two thymidines (2T; TT), the spacer regions in the amplifier probes are each a sequence of eight thymidines (8T; TTTTTTTT), and the spacer regions in the adapter probes are each a sequence of eight thymidines (8T; TTTTTTTT).

### A. Probes

In some aspects, the methods provided herein comprise contacting a biological sample with amplifier probes and reporter probes, and detecting complexes formed between the amplifier probes, the reporter probes, and one or more other probes or target nucleic acid molecules in the biological sample. In some embodiments, the methods further comprise contacting the biological sample with adapter probes and/or primary probes that form part of the complexes. Such probes and resulting hybridization complexes are described in further detail below.

### (i) Amplifier Probes

In some aspects, provided herein are amplifier probes. An amplifier probe comprises a recognition sequence and at least two reporter hybridization regions. The recognition sequence can be a sequence complementary to an amplifier probe hybridization region in an adapter probe, a primary probe, or a target nucleic acid (e.g., a nucleic acid analyte, a nucleic acid product such as a cDNA or rolling circle amplification product, or an oligonucleotide reporter associated with a non-nucleic acid analyte). In some embodiments, the recognition sequence is between about 15 and about 50 nucleotides in length, between about 16 and about 50 nucleotides in length, between about 20 and about 50 nucleotides in length, between about 20 and about 40 nucleotides in length, between about 16 and about 40 nucleotides in length, between about 16 and about 30 nucleotides in length, or between about 16 and about 25 nucleotides in length.

In some embodiments, the amplifier probe comprises 2, 3, 4, 5, 6, or more reporter hybridization regions. In some embodiments, the amplifier probe comprises between 2 and 20 reporter hybridization regions. In some embodiments, the amplifier probe comprises between 2 and 4, between 2 and 5, or between 2 and 6 reporter hybridization regions. In some embodiments, the amplifier probe comprises 3 reporter hybridization regions. In some embodiments, the at least two reporter hybridization regions are separated by a spacer region of at least 4 nucleotides in length. In some embodiments, the spacer region is at least 5, at least 6, at least 7, or at least 8 nucleotides in length. In some embodiments, the spacer region is no more than 10, no more than 9, no more than 8, or no more than 7 nucleotides in length. In some embodiments, the spacer region is between 4 and 10, between 4 and 9, between 4 and 8, or between 5 and 8 nucleotides in length. In some embodiments, the spacer region is between 4 and 20, between 4 and 15, between 4 and 10, between 8 and 20, or between 8 and 15 nucleotides in length. In some embodiments, each of the reporter hybridization regions has the same sequence (e.g., the reporter hybridization regions are copies of the same sequence to allow hybridization of multiple molecules of the same reporter probe to the amplifier probe).

In some embodiments, the amplifier probe comprises at least three reporter hybridization regions. In some embodiments, each of the reporter hybridization regions is separated by a spacer region. In some embodiments, each of the spacer regions is at least 4 nucleotides in length. In some embodiments, each of the spacer regions is at least 5, at least 6, at least 7, or at least 8 nucleotides in length. In some embodiments, each of the spacer regions is no more than 10, no more than 9, no more than 8, or no more than 7 nucleotides in length. In some embodiments, each of the spacer regions is between 4 and 10, between 4 and 9, between 4 and 8, or between 5 and 8 nucleotides in length. In some embodiments, each of the reporter hybridization regions has the same sequence (e.g., the reporter hybridization regions are copies of the same sequence to allow hybridization of multiple molecules of the same reporter probe to the amplifier probe). In some embodiments, each of the spacer regions has the same sequence. In some embodiments, the spacer regions have different sequences. In some embodiments, the spacer regions have random sequences. In some embodiments, the spacer region sequences are a sequence of adenines and/or thymidines.

In some embodiments, the amplifier probe comprises, from 5′ to 3′ or from 3′ to 5′, a first reporter hybridization region, a spacer region, and a second reporter hybridization region. In some embodiments, the amplifier probe comprises, from 5′ to 3′ or from 3′ to 5′, the recognition sequence, a first reporter hybridization region, a spacer region, and a second reporter hybridization region. In some embodiments, the first and second reporter hybridization regions are individually between 15 and 25 nucleotides in length, between 16 and 25 nucleotides in length, between 16 and 22 nucleotides in length, or between 16 and 20 nucleotides in length. In some embodiments, the reporter hybridization regions are individually about 16 nucleotides in length. The spacer region can be any of the spacer regions described herein (e.g., a nucleotide spacer region, such as a nucleotide sequence of between 4 and 8 nucleotides in length, or a spacer region comprising one or more organic non-nucleic acid spacer). In some embodiments, the amplifier probe further comprises a linker sequence of between 2 and 8 nucleotides in length between the recognition sequence and the first reporter hybridization region. In some embodiments, the linker sequence is a sequence of between 4 and 8 nucleotides in length or between 5 and 8 nucleotides in length. In some embodiments, the linker sequence is a sequence of about 2, 3, 4, 5, 6, 7, or 8 nucleotides in length.

In some embodiments, the amplifier probe comprises, from 5′ to 3′ or from 3′ to 5′, a first reporter hybridization region, a first spacer region, a second reporter hybridization region, a second spacer region, and a third reporter hybridization region. In some embodiments, the amplifier probe comprises, from 5′ to 3′ or from 3′ to 5′, the recognition sequence, a first reporter hybridization region, a first spacer region, a second reporter hybridization region, a second spacer region, and a third reporter hybridization region. In some embodiments, the first, second, and third reporter hybridization regions are individually between 15 and 25 nucleotides in length, between 16 and 25 nucleotides in length, between 16 and 22 nucleotides in length, or between 16 and 20 nucleotides in length. In some embodiments, the first, second, and third reporter hybridization regions are individually about 16 nucleotides in length. The first and second spacer regions can be any of the spacer regions described herein (e.g., a nucleotide spacer region, such as a nucleotide sequence of between 4 and 8 nucleotides in length, or a spacer region comprising one or more organic non-nucleic acid spacer). In some embodiments, the first and second spacer regions are the same (e.g., the same nucleotide sequence, such as a sequence of thymidines and/or adenines between 4 and 8 nucleotides in length). In some embodiments, the first and second spacer regions are different. In some embodiments, the amplifier probe further comprises a linker sequence of between 2 and 8 nucleotides in length between the recognition sequence and the first reporter hybridization region. In some embodiments, the linker sequence is a sequence of between 4 and 8 nucleotides in length or between 5 and 8 nucleotides in length. In some embodiments, the linker sequence is a sequence of about 2, 3, 4, 5, 6, 7, or 8 nucleotides in length.

In some embodiments, the spacer region is a random sequence of nucleotides. The random sequence can be a sequence of one or more degenerate bases. In some embodiments, each degenerate base can be any one of the naturally occurring nucleotides A, T, C, or G. In some embodiments, each degenerate base is selected from the group consisting of A and T. Thus, an amplifier probe comprising degenerate bases is actually a pool of amplifier probe probes having all possible sequence combinations or some random subset of random possible sequences at its degenerate position in the spacer regions. For example, if a DNA probe comprises a single degenerate base N, the probe is a set of four probes wherein the position N can be any of the naturally occurring bases (A, T, G, or C). In another example, if a DNA probe comprises a single degenerate base selected from A and T, the probe is a set of two probes wherein the degenerate position can be A or T.

In some embodiments, the spacer region is a sequence of thymidines and/or adenines. In some embodiments, the spacer region is a sequence of thymidines (e.g., 4T (TTTT), 5T (TTTTT), 6T (TTTTTT), 7T (TTTTTTT), or 8T (TTTTTTTT)). In some embodiments, the spacer region is a sequence of adenines (e.g., 4A (AAAA), 5A (AAAAA), 6A (AAAAA), 7A (AAAAAAA), or 8A (AAAAAAAA)). In some embodiment, the spacer region is a sequence of thymidines and adenines (e.g., any sequence of between 4 and 8 nucleotides in length composed on thymidines and adenines). In some embodiments, wherein the amplifier probe comprises multiple spacer regions (e.g., the amplifier probe comprises a first reporter hybridization region, a first spacer, a second reporter hybridization region, a second spacer, and a third reporter hybridization region), each spacer sequence is a sequence of thymidines and/or adenines. The spacer regions can be the same sequence or different sequences.

In some embodiments, the amplifier probe is between about 40 and 500 nucleotides in length. In some embodiments, the amplifier probe is between about 50 and about 500 nucleotides in length. In some embodiments, the amplifier probe is between any of about 40 and about 60, about 45 and about 60, about 50 and about 60, or about 45 and about 55 nucleotides in length.

In some embodiments, the spacer region is a region comprising one or more non-nucleic acid spacers. In some embodiments, non-nucleic acid spacers are organic non-nucleic acid spacers. In some embodiments, the spacer region comprises one organic spacer. In some embodiments, the spacer region comprises two or more organic non-nucleic acid spacers (e.g., 2, 3, 4, or more organic non-nucleic acid spacers). Exemplary organic non-nucleic acid spacers are include but are not limited to a triethylene glycol spacer, a C3 spacer phosphoramidite, a hexanediol, an 18-atom hexa-ethyleneglycol spacer, a glycol linker, a carbon chain, and a polyethylene glycol (PEG) spacer. In some embodiments, the spacer region comprises a combination of any of the aforementioned organic non-nucleic acid spacers. In some embodiments, the amplifier probe comprising the organic non-nucleotide spacer is prepared using standard desalting or cartridge purification. In some embodiments, the amplifier probe is purified using HPLC purification.

In some embodiments, the one or more organic spacers comprise an 18-atom hexa-ethyleneglycol spacer. Hexaethylene glycol is an example of as polyethylene glycol spacer that can be placed internally in a nucleic acid molecule. The 18 atom spacer can be placed at a 5′ end, a 3′ end, or internally. When used in a spacer region between two reporter hybridization regions, the spacer is placed internally in the nucleic acid molecule. In some embodiments, the spacer region comprises multiple PEG spacers, such as multiple hexaethylene glycol spacers.

In some embodiments, the organic non-nucleic acid spacer comprises a carbon chain. In some embodiments, the organic non-nucleic acid spacer is a C3, C6, or C12 spacer. In some embodiments, the spacer region is no more than about 10 nm in length. In some embodiments, the spacer region is at least 2.5, 3, 4, or 5 nm in length. In some embodiments of the methods provided herein, the signal from the detectable label of the reporter probe detected in (b) is brighter than a control signal of a reporter probe having the same detectable label hybridized to a control amplifier probe that comprises a spacer region of less than 2 nm in length between each of at least two reporter hybridization regions in the control amplifier probe. In some embodiments of the methods provided herein, the signal-to-noise ratio from the detectable label of the reporter probe detected in (b) is greater than a control the signal-to-noise ratio for a reporter probe having the same detectable label hybridized to a control amplifier probe that comprises a spacer region of less than 2 nm in length between each of at least two reporter hybridization regions in the control amplifier probe. In some embodiments, the signal-to-noise ratio from the detectable label of the reporter probe detected in (b) is at least 10%, at least 20%, at least 30%, at least 40%, at least 50%, or at least 55% higher than the signal-to-noise ratio for the reporter probe having the same detectable label hybridized to the control amplifier probe.

### (ii) Adapter Probes

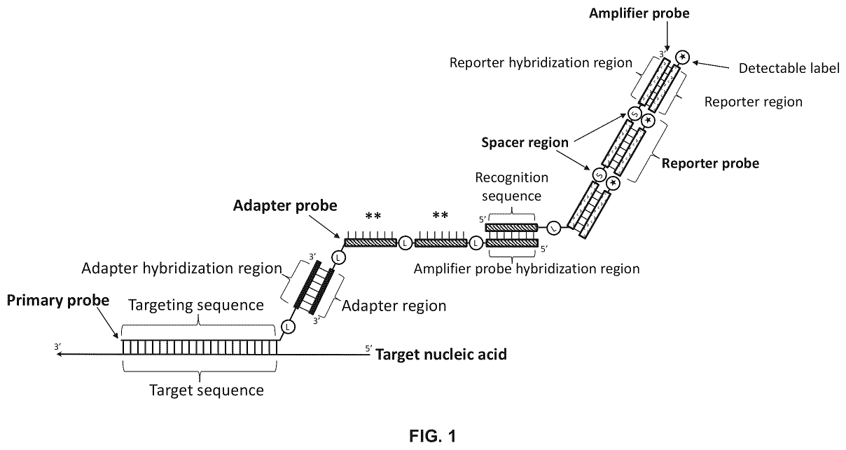

In some embodiments, also provided herein are adapter probes. In some cases, an adapter probe binds to an adapter hybridization region in a primary probe (e.g., as illustrated in FIG. 1), and the adapter probe comprises one or more amplifier probe hybridization regions for binding of amplifier probes. In some instances, the adapter probe can provide for additional signal amplification by providing multiple adapter probe hybridization regions, thus allowing multiple amplifier probes to hybridize to the adapter probe (e.g., in a branched configuration). FIG. 1 illustrates an exemplary adapter probe comprising three amplifier probe hybridization regions. In the illustration of FIG. 1, the recognition sequence of the amplifier probe is complementary to an amplifier probe hybridization region in an adapter probe. For simplicity, a single amplifier probe is shown hybridized to the adapter probe. As indicated by the asterisks (**) above the other two amplifier probe hybridization regions, amplifier probes can similarly be bound to the other amplifier probe hybridization regions (not shown for simplicity).

In some embodiments, the adapter probe comprises multiple copies of the amplifier probe hybridization region. For example, FIG. 1 illustrates an adapter probe comprising 3 amplifier probe hybridization regions. In some embodiments, the adapter probe comprises 2, 3, 4, 5, 6, or more amplifier probe hybridization regions. In some embodiments, the adapter probe comprises between 2 and 20 amplifier probe hybridization regions. In some embodiments, the adapter probe comprises between 2 and 4, between 2 and 5, or between 2 and 6 amplifier probe hybridization regions. In some embodiments, the adapter probe comprises 3 amplifier probe hybridization regions. In some embodiments, each of the amplifier probe hybridization regions has the same sequence (e.g., the amplifier probe hybridization regions are copies of the same sequence to allow hybridization of multiple molecules of the same amplifier probe to the adapter probe). In some embodiments, the amplifier probe hybridization regions are separated by a linker region of at least 2 nucleotides in length. FIG. 1, for example, illustrates an exemplary adapter probe comprising three amplifier probe hybridization regions separated by linker regions (depicted as an “L”). In some embodiments, the amplifier probe hybridization regions are separated by a linker region of at least 4 nucleotides in length. In some embodiments, the linker region is at least 5, at least 6, at least 7, or at least 8 nucleotides in length. In some embodiments, the linker region is no more than 10, no more than 9, no more than 8, or no more than 7 nucleotides in length. In some embodiments, the linker region is between 4 and 10, between 4 and 9, between 4 and 8, or between 5 and 8 nucleotides in length. In the case of an adapter probe comprising two or more linker regions (e.g., two or more linker regions), the linker regions can be the same or different.

In some embodiments, the detected complex comprises at least 2 amplifier probes hybridized to the adapter probe. In some embodiments, the detected complex comprises at least 2, 3, 4, 5, 6, or more amplifier probes hybridized to the adapter probe. In some embodiments, the detected complex comprises between 2 and 20 amplifier probes hybridized to the adapter probe. In some embodiments, the detected complex comprises at least 2, 3, 4, 5, 6, or more reporter probes hybridized to each amplifier probe. In some embodiments, the detected complex comprises at least 2, 3, 4, 5, 6, or more adapter probes bound to the primary probe. In some embodiments, the detected complex comprises at least 10, at least 20, at least 30, or at least 40 reporter probes bound in the complex. In some embodiments, the detected signal is the signal of at least 10, at least 20, at least 30, or at least 40 reporter probes bound in the complex.

In some embodiments, the adapter probe comprises, from 5′ to 3′ or from 3′ to 5′, a first amplifier probe hybridization region, a linker region, and a second amplifier probe hybridization region. In some embodiments, the adapter probe comprises, from 5′ to 3′ or from 3′ to 5′, the adapter region, a first amplifier probe hybridization region, a linker region, and a second amplifier probe hybridization region. In some embodiments, the first and second amplifier probe hybridization regions are individually between 15 and 25 nucleotides in length, between 16 and 25 nucleotides in length, between 16 and 22 nucleotides in length, or between 16 and 20 nucleotides in length. In some embodiments, the amplifier probe hybridization regions are individually about 16 nucleotides in length. The linker region can be any of the linker regions described herein (e.g., a nucleotide linker region, such as a nucleotide sequence of between 4 and 8 nucleotides in length, or a linker region comprising one or more organic non-nucleic acid spacer). In some embodiments, the adapter probe further comprises a linker sequence of between 2 and 8 nucleotides in length between the adapter region and the first amplifier probe hybridization region. In some embodiments, the linker sequence is a sequence of between 4 and 8 nucleotides in length or between 5 and 8 nucleotides in length. In some embodiments, the linker sequence is a sequence of about 2, 3, 4, 5, 6, 7, or 8 nucleotides in length.

In some embodiments, the adapter probe comprises, from 5′ to 3′ or from 3′ to 5′, a first amplifier probe hybridization region, a first linker region, a second amplifier probe hybridization region, a second linker region, and a third amplifier probe hybridization region. In some embodiments, the adapter probe comprises, from 5′ to 3′ or from 3′ to 5′, the adapter region, a first amplifier probe hybridization region, a first linker region, a second amplifier probe hybridization region, a second linker region, and a third amplifier probe hybridization region. In some embodiments, the first, second, and third amplifier probe hybridization regions are individually between 15 and 25 nucleotides in length, between 16 and 25 nucleotides in length, between 16 and 22 nucleotides in length, or between 16 and 20 nucleotides in length. In some embodiments, the first, second, and third amplifier probe hybridization regions are individually about 16 nucleotides in length. The first and second linker regions can be any of the linker regions described herein (e.g., a nucleotide linker region, such as a nucleotide sequence of between 2 and 10 nucleotides in length, or a linker region comprising one or more organic non-nucleic acid spacer). In some embodiments, the first and second linker regions are the same (e.g., the same nucleotide sequence, such as a sequence of thymidines and/or adenines between 2 and 10 nucleotides in length). In some embodiments, the first and second linker regions are different. In some embodiments, the adapter probe further comprises a linker sequence of between 2 and 8 nucleotides in length between the adapter region and the first amplifier probe hybridization region. In some embodiments, the linker sequence is a sequence of between 4 and 8 nucleotides in length or between 5 and 8 nucleotides in length. In some embodiments, the linker sequence is a sequence of about 2, 3, 4, 5, 6, 7, or 8 nucleotides in length.

In some embodiments, the linker region is a random sequence of nucleotides. The random sequence can be a sequence of one or more degenerate bases. In some embodiments, each degenerate base can be any one of the naturally occurring nucleotides A, T, C, or G. In some embodiments, each degenerate base is selected from the group consisting of A and T. Thus, an adapter probe comprising degenerate bases is actually a pool of adapter probe probes having all possible sequence combinations or some random subset of random possible sequences at its degenerate position in the linker regions. For example, if a DNA probe comprises a single degenerate base N, the probe is a set of four probes wherein the position N can be any of the naturally occurring bases (A, T, G, or C). In another example, if a DNA probe comprises a single degenerate base selected from A and T, the probe is a set of two probes wherein the degenerate position can be A or T.

In some embodiments, the linker region is a sequence of thymidines and/or adenines. In some embodiments, the linker region is a sequence of thymidines (e.g., 2T, 4T (TTTT), 5T (TTTTT), 6T (TTTTTT), 7T (TTTTTTT), or 8T (TTTTTTTT)). In some embodiments, the linker region is a sequence of adenines (e.g., 2A, 4A (AAAA), 5A (AAAAA), 6A (AAAAA), 7A (AAAAAAA), or 8A (AAAAAAAA)). In some embodiment, the linker region is a sequence of thymidines and adenines (e.g., any sequence of between 2 and 10 nucleotides in length composed on thymidines and adenines). In some embodiments, wherein the adapter probe comprises multiple linker regions (e.g., the adapter probe comprises a first amplifier probe hybridization region, a first spacer, a second amplifier probe hybridization region, a second spacer, and a third amplifier probe hybridization region), each spacer sequence is a sequence of thymidines and/or adenines. The linker regions can be the same sequence or different sequences.

In some embodiments, the linker region is a region comprising one or more non-nucleic acid spacers. In some embodiments, non-nucleic acid spacers are organic non-nucleic acid spacers. In some embodiments, the linker region comprises one organic spacer. In some embodiments, the linker region comprises two or more organic non-nucleic acid spacers (e.g., 2, 3, 4, or more organic non-nucleic acid spacers). Exemplary organic non-nucleic acid spacers are include but are not limited to a triethylene glycol spacer, a C3 spacer phosphoramidite, a hexanediol, an 18-atom hexa-ethyleneglycol spacer, a glycol linker, a carbon chain, and a polyethylene glycol (PEG) spacer. In some embodiments, the linker region comprises a combination of any of the aforementioned organic non-nucleic acid spacers. In some embodiments, the adapter probe comprising the organic non-nucleotide spacer is prepared using standard desalting or cartridge purification. In some embodiments, the adapter probe is purified using HPLC purification.

In some embodiments, the one or more organic spacers comprise an 18-atom hexa-ethyleneglycol spacer. Hexaethylene glycol is an example of as polyethylene glycol spacer that can be placed internally in a nucleic acid molecule. The 18 atom spacer can be placed at a 5′ end, a 3′ end, or internally. When used in a linker region between two amplifier probe hybridization regions, the spacer is placed internally in the nucleic acid molecule. In some embodiments, the linker region comprises multiple PEG spacers, such as multiple hexaethylene glycol spacers.

In some embodiments, the organic non-nucleic acid spacer comprises a carbon chain. In some embodiments, the organic non-nucleic acid spacer is a C3, C6, or C12 spacer. In some embodiments, the linker region is no more than about 10 nm in length. In some embodiments, the linker region is at least 2.5, 3, 4, or 5 nm in length.

In some embodiments, the adapter probe is between about 40 and 500 nucleotides in length. In some embodiments, the adapter probe is between about 50 and about 500 nucleotides in length. In some embodiments, the adapter probe is between any of about 40 and about 60, about 45 and about 60, about 50 and about 60, or about 45 and about 55 nucleotides in length.

(iii) Primary Probes

In some embodiments, also provided herein are primary probes. In some cases, an adapter probe binds to an adapter hybridization region in a primary probe (e.g., as illustrated in FIG. 1). In some embodiments, the amplifier probe hybridizes directly to the primary probe, without an adapter probe intermediary. In some embodiments, the primary probe comprises a targeting sequence that is complementary to a target sequence in a target nucleic acid. The target nucleic acid can be any of the target nucleic acid molecules described herein, such as an endogenous cellular nucleic acid analyte (e.g., RNAs), a nucleic acid product such as a cDNA or rolling circle amplification product provided as a proxy of an analyte (e.g., a rolling circle amplification product produced from a circular or circularized probe or probe set that binds directly or indirectly to an analyte or labeling agent), or an oligonucleotide reporter associated with a non-nucleic acid analyte such as a protein.

In some embodiments, the targeting sequence is between about 15 and about 50 nucleotides in length, between about 16 and about 50 nucleotides in length, between about 20 and about 50 nucleotides in length, between about 20 and about 40 nucleotides in length, between about 16 and about 40 nucleotides in length, between about 16 and about 30 nucleotides in length, or between about 16 and about 25 nucleotides in length.

FIG. 1 illustrates an exemplary complex wherein the primary probe binds to an adapter probe, which binds to the amplifier probe. In some embodiments, the primary probe comprises an overhang region comprising the adapter hybridization region. The overhang region can be at the 5′ end or the 3′ end of the primary probe. In some embodiments, the primary probe comprises a first overhang region and a second overhang region (e.g., a 5′ overhang region and a 3′ overhang region). In some embodiments, the primary probe comprises 2, 3, 4, 5, 6, or more adapter hybridization regions in one or more overhang regions of the primary probe. For example, in some cases the primary probe comprises a first overhang region comprising a first adapter hybridization region and a second adapter hybridization region, wherein the adapter hybridization regions are separated by a linker region. In some embodiments, the first adapter hybridization region is separated from the targeting sequence by a linker region. In some embodiments, the primary probe comprises a first overhang region comprising a first adapter hybridization region, a second adapter hybridization region, and a third adapter hybridization region, wherein the adapter hybridization regions are separated by a linker region.

In some embodiments, the amplifier probe(s) bind to the primary probe. In some embodiments, the primary probe comprises an overhang region comprising the amplifier probe hybridization region. The overhang region can be at the 5′ end or the 3′ end of the primary probe. In some embodiments, the primary probe comprises a first overhang region and a second overhang region (e.g., a 5′ overhang region and a 3′ overhang region). In some embodiments, the primary probe comprises 2, 3, 4, 5, 6, or more amplifier probe hybridization regions in one or more overhang regions of the primary probe. For example, in some cases the primary probe comprises a first overhang region comprising a first amplifier probe hybridization region and a second amplifier probe hybridization region, wherein the amplifier probe hybridization regions are separated by a linker region. In some embodiments, the first amplifier probe hybridization region is separated from the targeting sequence by a linker region. In some embodiments, the primary probe comprises a first overhang region comprising a first amplifier probe hybridization region, a second amplifier probe hybridization region, and a third amplifier probe hybridization region, wherein the amplifier probe hybridization regions are separated by a linker region.

In some embodiments, the first, second, and/or third amplifier probe hybridization regions are individually between 15 and 25 nucleotides in length, between 16 and 25 nucleotides in length, between 16 and 22 nucleotides in length, or between 16 and 20 nucleotides in length. In some embodiments, the first, second, and/or third amplifier probe hybridization regions are individually about 16 nucleotides in length. The linker regions can be any of the linker regions described herein (e.g., a nucleotide linker region, such as a nucleotide sequence of between 2 and 10 nucleotides in length, or a linker region comprising one or more organic non-nucleic acid spacer). In some embodiments, the first and second linker regions are the same (e.g., the same nucleotide sequence, such as a sequence of thymidines and/or adenines between 2 and 10 nucleotides in length). In some embodiments, the first and second linker regions are different. In some embodiments, the adapter probe further comprises a linker sequence of between 2 and 8 nucleotides in length between the adapter region and the first amplifier probe hybridization region. In some embodiments, the linker sequence is a sequence of between 4 and 8 nucleotides in length or between 5 and 8 nucleotides in length. In some embodiments, the linker sequence is a sequence of about 2, 3, 4, 5, 6, 7, or 8 nucleotides in length.

In some embodiments, the linker region is a sequence of thymidines and/or adenines. In some embodiments, the linker region is a sequence of thymidines (e.g., 2T, 4T (TTTT), 5T (TTTTT), 6T (TTTTTT), 7T (TTTTTTT), or 8T (TTTTTTTT)). In some embodiments, the linker region is a sequence of adenines (e.g., 2A, 4A (AAAA), 5A (AAAAA), 6A (AAAAA), 7A (AAAAAAA), or 8A (AAAAAAAA)). In some embodiment, the linker region is a sequence of thymidines and adenines (e.g., any sequence of between 2 and 10 nucleotides in length composed on thymidines and adenines). In some embodiments, wherein the adapter probe comprises multiple linker regions (e.g., the adapter probe comprises a first amplifier probe hybridization region, a first spacer, a second amplifier probe hybridization region, a second spacer, and a third amplifier probe hybridization region), each spacer sequence is a sequence of thymidines and/or adenines. The linker regions can be the same sequence or different sequences.

In some embodiments, the linker region is a region comprising one or more non-nucleic acid spacers. In some embodiments, non-nucleic acid spacers are organic non-nucleic acid spacers. In some embodiments, the linker region comprises one organic spacer. In some embodiments, the linker region comprises two or more organic non-nucleic acid spacers (e.g., 2, 3, 4, or more organic non-nucleic acid spacers). Exemplary organic non-nucleic acid spacers are include but are not limited to a triethylene glycol spacer, a C3 spacer phosphoramidite, a hexanediol, an 18-atom hexa-ethyleneglycol spacer, a glycol linker, a carbon chain, and a polyethylene glycol (PEG) spacer. In some embodiments, the linker region comprises a combination of any of the aforementioned organic non-nucleic acid spacers. In some embodiments, the adapter probe comprising the organic non-nucleotide spacer is prepared using standard desalting or cartridge purification. In some embodiments, the adapter probe is purified using HPLC purification.

In some embodiments, the one or more organic spacers comprise an 18-atom hexa-ethyleneglycol spacer. Hexaethylene glycol is an example of as polyethylene glycol spacer that can be placed internally in a nucleic acid molecule. The 18 atom spacer can be placed at a 5′ end, a 3′ end, or internally. When used in a linker region between two amplifier probe hybridization regions, the spacer is placed internally in the nucleic acid molecule. In some embodiments, the linker region comprises multiple PEG spacers, such as multiple hexaethylene glycol spacers.

In some embodiments, the organic non-nucleic acid spacer comprises a carbon chain. In some embodiments, the organic non-nucleic acid spacer is a C3, C6, or C12 spacer. In some embodiments, the linker region is no more than about 10 nm in length. In some embodiments, the linker region is at least 2.5, 3, 4, or 5 nm in length.

In some embodiments, the primary probe is between about 40 and 500 nucleotides in length. In some embodiments, the primary probe is between about 50 and about 500 nucleotides in length. In some embodiments, the primary probe is between any of about 40 and about 60, about 45 and about 60, about 50 and about 60, or about 45 and about 55 nucleotides in length.

### (iv) Reporter Probes

In some aspects, the methods provided herein comprise hybridizing reporter probes to amplifier probes comprising multiple reporter hybridization regions, wherein the reporter hybridization regions are separated by one or more spacer regions. In some embodiments, the reporter hybridization regions are separated by spacer regions of at least 4 nucleotides in length. In some embodiments, the reporter hybridization regions are separated by a spacer region comprising one or more non-nucleotide spacers.

A reporter probe herein comprises a detectable label and a reporter region. The reporter region is complementary to a reporter hybridization sequence in the amplifier probe, and hybridizes to the amplifier probe. In some embodiments, the reporter probes are individually between 15 and 25 nucleotides in length, between 15 and 30 nucleotides in length, between 15 and 40 nucleotides in length, or between 20 and 40 nucleotides in length. In some embodiments, the reporter region is between 15 and 25 nucleotides in length, between 15 and 20 nucleotides in length, between 16 and 25 nucleotides in length, between 16 and 22 nucleotides in length, or between 20 and 25 nucleotides in length. In some embodiments, the reporter region is no more than any one of 16, 17, 18, 19, 20, 22, or 25 nucleotides in length.

In some embodiments, the detectable label is a label that is measured and quantitated. The detectable label can comprise a directly or indirectly detectable moiety, e.g., any fluorophores, radioactive isotopes, fluorescers, chemiluminescers, enzymes, enzyme substrates, enzyme cofactors, enzyme inhibitors, chromophores, dyes, metal ions, metal sols, ligands (e.g., biotin or haptens) and the like.

In some embodiments, the detectable label is a fluorophore. A fluorophore can comprise a substance or a portion thereof that is capable of exhibiting fluorescence in the detectable range. Particular examples of labels that may be used in accordance with the provided embodiments comprise, but are not limited to phycoerythrin, Alexa dyes, fluorescein, YPet, CyPet, Cascade blue, allophycocyanin, Cy3, Cy5, Cy7, rhodamine, dansyl, umbelliferone, Texas red, luminol, acradimum esters, biotin, green fluorescent protein (GFP), enhanced green fluorescent protein (EGFP), yellow fluorescent protein (YFP), enhanced yellow fluorescent protein (EYFP), blue fluorescent protein (BFP), red fluorescent protein (RFP), firefly luciferase, Renilla luciferase, NADPH, beta-galactosidase, horseradish peroxidase, glucose oxidase, alkaline phosphatase, chloramphenical acetyl transferase, and urease.

Fluorescence detection in tissue samples can often be hindered by the presence of strong background fluorescence. Background fluorescence can include autofluorescence (that can arise from a variety of sources, including aldehyde fixation, extracellular matrix components, red blood cells, lipofuscin, and the like), as opposed to the desired immunofluorescence from the fluorescently labeled antibodies or probes. Tissue autofluorescence can lead to difficulties in distinguishing the signals due to fluorescent antibodies or probes from the general background. In some embodiments, a method disclosed herein utilizes one or more agents to reduce tissue autofluorescence, for example, Autofluorescence Eliminator (Sigma/EMD Millipore), TrueBlack Lipofuscin Autofluorescence Quencher (Biotium), MaxBlock Autofluorescence Reducing Reagent Kit (MaxVision Biosciences), and/or a very intense black dye (e.g., Sudan Black, or comparable dark chromophore).

Examples of detectable labels comprise but are not limited to various radioactive moieties, enzymes, prosthetic groups, fluorescent markers, luminescent markers, bioluminescent markers, metal particles, protein-protein binding pairs and protein-antibody binding pairs. Examples of fluorescent proteins comprise, but are not limited to, yellow fluorescent protein (YFP), green fluorescence protein (GFP), cyan fluorescence protein (CFP), umbelliferone, fluorescein, fluorescein isothiocyanate, rhodamine, dichlorotriazinylamine fluorescein, dansyl chloride and phycoerythrin.

Examples of bioluminescent markers comprise, but are not limited to, luciferase (e.g., bacterial, firefly and click beetle), luciferin, aequorin and the like. Examples of enzyme systems having visually detectable signals comprise, but are not limited to, galactosidases, glucorimidases, phosphatases, peroxidases and cholinesterases. Identifiable markers also comprise radioactive compounds such as ¹²⁵I, ³⁵S, ¹⁴C, or ³H. Identifiable markers are commercially available from a variety of sources.

Examples of fluorescent labels and nucleotides and/or polynucleotides conjugated to such fluorescent labels comprise those described in, for example, Hoagland, Handbook of Fluorescent Probes and Research Chemicals, Ninth Edition (Molecular Probes, Inc., Eugene, 2002); Keller and Manak, DNA Probes, 2nd Edition (Stockton Press, New York, 1993); Eckstein, editor, Oligonucleotides and Analogues: A Practical Approach (IRL Press, Oxford, 1991); and Wetmur, Critical Reviews in Biochemistry and Molecular Biology, 26:227-259 (1991). In some embodiments, exemplary techniques and methods methodologies applicable to the provided embodiments comprise those described in, for example, U.S. Pat. Nos. 4,757,141, 5,151,507 and 5,091,519, all of which are herein incorporated by reference in their entireties. In some embodiments, one or more fluorescent dyes are used as labels for labeled target sequences, for example, as described in U.S. Pat. No. 5,188,934 (4,7-dichlorofluorescein dyes); U.S. Pat. No. 5,366,860 (spectrally resolvable rhodamine dyes); U.S. Pat. No. 5,847,162 (4,7-dichlororhodamine dyes); U.S. Pat. No. 4,318,846 (ether-substituted fluorescein dyes); U.S. Pat. No. 5,800,996 (energy transfer dyes); U.S. Pat. No. 5,066,580 (xanthine dyes); and U.S. Pat. No. 5,688,648 (energy transfer dyes), all of which are herein incorporated by reference in their entireties. Labeling can also be carried out with quantum dots, as described in U.S. Pat. Nos. 6,322,901, 6,576,291, 6,423,551, 6,251,303, 6,319,426, 6,426,513, 6,444,143, 5,990,479, 6,207,392, US 2002/0045045 and US 2003/0017264, all of which are herein incorporated by reference in their entireties. In some embodiments, a fluorescent label comprises a signaling moiety that conveys information through the fluorescent absorption and/or emission properties of one or more molecules. Exemplary fluorescent properties comprise fluorescence intensity, fluorescence lifetime, emission spectrum characteristics and energy transfer.

Examples of commercially available fluorescent nucleotide analogues readily incorporated into nucleotide and/or polynucleotide sequences comprise, but are not limited to, Cy3-dCTP, Cy3-dUTP, Cy5-dCTP, Cy5-dUTP (Amersham Biosciences, Piscataway, N.J.), fluorescein-12-dUTP, tetramethylrhodamine-6-dUTP, TEXAS RED™-5-dUTP, CASCADE BLUE™-7-dUTP, BODIPY TMFL-14-dUTP, BODIPY TMR-14-dUTP, BODIPY TMTR-14-dUTP, RHOD AMINE GREEN™-5-dUTP, OREGON GREENR™ 488-5-dUTP, TEXAS RED™-12-dUTP, BODIPY™ 630/650-14-dUTP, BODIPY™ 650/665-14-dUTP, ALEXA FLUOR™ 488-5-dUTP, ALEXA FLUOR™ 532-5-dUTP, ALEXA FLUOR™ 568-5-dUTP, ALEXA FLUOR™ 594-5-dUTP, ALEXA FLUOR™ 546-14-dUTP, fluorescein-12-UTP, tetramethylrhodamine-6-UTP, TEXAS RED™-5-UTP, mCherry, CASCADE BLUE™-7-UTP, BODIPY™ FL-14-UTP, BODIPY TMR-14-UTP, BODIPY™ TR-14-UTP, RHOD AMINE GREEN™-5-UTP, ALEXA FLUOR™ 488-5-UTP, and ALEXA FLUOR™ 546-14-UTP (Molecular Probes, Inc. Eugene, Oreg.). Methods are known for custom synthesis of nucleotides having other fluorophores (See, Henegariu et al. (2000) Nature Biotechnol. 18:345).

Other fluorophores available for post-synthetic attachment comprise, but are not limited to, ALEXA FLUOR™ 350, ALEXA FLUOR™ 532, ALEXA FLUOR™ 546, ALEXA FLUOR™ 568, ALEXA FLUOR™ 594, ALEXA FLUOR™ 647, BODIPY 493/503, BODIPY FL, BODIPY R6G, BODIPY 530/550, BODIPY TMR, BODIPY 558/568, BODIPY 558/568, BODIPY 564/570, BODIPY 576/589, BODIPY 581/591, BODIPY 630/650, BODIPY 650/665, Cascade Blue, Cascade Yellow, Dansyl, lissamine rhodamine B, Marina Blue, Oregon Green 488, Oregon Green 514, Pacific Blue, rhodamine 6G, rhodamine green, rhodamine red, tetramethyl rhodamine, Texas Red (available from Molecular Probes, Inc., Eugene, Oreg.), Cy2, Cy3.5, Cy5.5, and Cy7 (Amersham Biosciences, Piscataway, N.J.). FRET tandem fluorophores may also be used, comprising, but not limited to, PerCP-Cy5.5, PE-Cy5, PE-Cy5.5, PE-Cy7, PE-Texas Red, APC-Cy7, PE-Alexa dyes (610, 647, 680), and APC-Alexa dyes.

In some cases, metallic silver or gold particles may be used to enhance signal from fluorescently labeled nucleotide and/or polynucleotide sequences (Lakowicz et al. (2003) Bio Techniques 34:62).

Biotin, or a derivative thereof, may also be used as a label on a nucleotide and/or a polynucleotide sequence, and subsequently bound by a detectably labeled avidin/streptavidin derivative (e.g., phycoerythrin-conjugated streptavidin), or a detectably labeled anti-biotin antibody. Digoxigenin may be incorporated as a label and subsequently bound by a detectably labeled anti-digoxigenin antibody (e.g., fluoresceinated anti-digoxigenin). An aminoallyl-dUTP residue may be incorporated into a polynucleotide sequence and subsequently coupled to an N-hydroxy succinimide (NHS) derivatized fluorescent dye. In general, any member of a conjugate pair may be incorporated into a detection polynucleotide provided that a detectably labeled conjugate partner can be bound to permit detection.

Other suitable labels for a polynucleotide sequence may comprise fluorescein (FAM), digoxigenin, dinitrophenol (DNP), dansyl, biotin, bromodeoxyuridine (BrdU), hexahistidine (6×His), and phosphor-amino acids (e.g., P-tyr, P-ser, P-thr). In some embodiments the following hapten/antibody pairs are used for detection, in which each of the antibodies is derivatized with a detectable label: biotin/a-biotin, digoxigenin/a-digoxigenin, dinitrophenol (DNP)/a-DNP, 5-Carboxyfluorescein (FAM)/a-FAM.

In some embodiments, a nucleotide and/or a oligonucleotide sequence is indirectly labeled, especially with a hapten that is then bound by a capture agent, e.g., as disclosed in U.S. Pat. Nos. 5,344,757, 5,702,888, 5,354,657, 5,198,537 and 4,849,336, and 5,073,562, all of which are herein incorporated by reference in their entireties. Many different hapten-capture agent pairs are available for use. Exemplary haptens comprise, but are not limited to, biotin, des-biotin and other derivatives, dinitrophenol, dansyl, fluorescein, Cy5, and digoxigenin. For biotin, a capture agent may be avidin, streptavidin, or antibodies. Antibodies may be used as capture agents for the other haptens (many dye-antibody pairs being commercially available, e.g., Molecular Probes, Eugene, Oreg.).

In some embodiments, the detectable label is a first detectable label, and the reporter probe further comprises a second detectable label. In some embodiments, the first detectable label and the second detectable label are different. In some embodiments, detecting a complex comprising the reporter probe in the biological sample comprises detecting a first signal from the first detectable label and detecting a second signal from the second detectable label. In some embodiments, the first detectable label is at the 5′ end of the reporter probe and the second detectable label is at the 3′ end of the reporter probe.

In some embodiments, the reporter probe comprises a flexible linker between the reporter region and the first detectable label. In some embodiments, the flexible linker is a nucleotide sequence of between 1 and 10 nucleotides in length (e.g., between any of 2 and 10, 2 and 8, or 4 and 8 nucleotides in length). In some embodiments, the flexible linker is a non-nucleic acid linker. In some embodiments, the detectable label is linked to the reporter region by a disulfide.

In some embodiments, the detectable label is a fluorophore having an excitation peak between 480 nm and 500 nm. In some embodiments, the detectable label is a fluorophore having an excitation peak between 520 nm and 540 nm. In some embodiments, the detectable label is a fluorophore having an excitation peak between 590 nm and 600 nm. In some embodiments, the detectable label is a fluorophore having an excitation peak between 640 nm and 660 nm.

### B. Detection and Analysis

In some aspects, the provided methods involve analyzing, e.g., detecting or determining, one or more sequences present in a target nucleic acid molecule in the biological sample. In some embodiments, analyzing (e.g., detecting or determining) the one or more sequences in the target nucleic acid molecules comprises detecting the complex comprising the amplifier probe and the reporter probes. In some embodiments, the detecting is performed at one or more locations in the biological sample. In some embodiments, the locations are the locations of RNA transcripts in the biological sample. In some embodiments, the locations are the locations at which the probes or probe sets hybridize to the RNA transcripts in the biological sample, and are optionally ligated and amplified by rolling circle amplification.

In some embodiments, the detecting comprises a plurality of repeated cycles of probe complex formation and removal. For example, in some embodiments the detecting comprises a plurality of repeated cycles of binding and removal of probes (e.g., amplifier probes and/or reporter probes) to the primary probe or probe set hybridized to the target nucleic acid, optionally wherein the amplifier probes bind to the primary probe indirectly (via an adapter probe). In some embodiments, the detecting comprises a plurality of repeated cycles of binding and removal of probes (e.g., amplifier probes and reporter probes) to the target nucleic acid, optionally wherein the amplifier probes bind to the target nucleic acid indirectly (e.g., via an adapter probe and/or a primary probe). In some embodiments, the target nucleic acid is a nucleic acid analyte. In some embodiments, the target nucleic acid is a probe or a product provided as a proxy for a nucleic acid analyte (e.g., a rolling circle amplification product generated from a circular or circularized probe that binds directly or indirectly to the nucleic acid analyte). In some embodiments, the target nucleic acid is a nucleic acid molecule provided as a proxy for a non-nucleic acid analyte (e.g., an oligonucleotide reporter in a labeling agent that binds to the non-nucleic acid analyte, or a product such as a rolling circle amplification product associated with the oligonucleotide reporter). In some embodiments, the detecting comprises a plurality of repeated cycles of binding and removal of probes (e.g., amplifier probes and/or reporter probes) to the target nucleic acid, wherein the amplifier probes bind to an oligonucleotide reporter conjugated to a labeling agent (e.g., an antibody)). In some embodiments, after each round of detection, amplifier probes and reporter probes are removed to allow a different amplifier probe to bind in the next cycle.

Methods for binding and identifying a target nucleic acid that uses various probes or oligonucleotides have been described in, e.g., US2003/0013091, US2007/0166708, US2010/0015607, US2010/0261026, US2010/0262374, US2010/0112710, US2010/0047924, and US2014/0371088, each of which is incorporated herein by reference in its entirety. Detectably-labeled probes can be useful for detecting multiple target nucleic acids and be detected in one or more hybridization cycles (e.g., sequential hybridization assays, or sequencing by hybridization).

In some embodiments, the detecting can comprise binding an amplifier probe directly or indirectly (e.g., via an adapter) to the primary probe or probe set, binding a detectably labeled reporter probe to the at reporter hybridization regions of the amplifier probe, and detecting a signal associated with the detectably labeled reporter probe. In some embodiments, the method can comprise performing one or more wash steps to remove unbound and/or nonspecifically bound amplifier and/or reporter probes.

In some aspects, the detecting comprises imaging the biological sample. In some aspects, the detecting comprises performing microscopy, scanning mass spectrometry or other imaging techniques described herein. In such aspects, the detecting comprises determining a signal, e.g., a fluorescent signal.

In some aspects, the detection (comprising imaging) is carried out using any of a number of different types of microscopy, e.g., confocal microscopy, two-photon microscopy, light-field microscopy, intact tissue expansion microscopy, and/or CLARITY™-optimized light sheet microscopy (COLM).

In some embodiments, fluorescence microscopy is used for detection and imaging of the detection probe. In some aspects, a fluorescence microscope is an optical microscope that uses fluorescence and phosphorescence instead of, or in addition to, reflection and absorption to study properties of organic or inorganic substances. In fluorescence microscopy, a sample is illuminated with light of a wavelength which excites fluorescence in the sample. The fluoresced light, which is usually at a longer wavelength than the illumination, is then imaged through a microscope objective. Two filters may be used in this technique; an illumination (or excitation) filter which ensures the illumination is near monochromatic and at the correct wavelength, and a second emission (or barrier) filter which ensures none of the excitation light source reaches the detector. Alternatively, these functions may both be accomplished by a single dichroic filter. The fluorescence microscope can be any microscope that uses fluorescence to generate an image, whether it is a more simple set up like an epifluorescence microscope, or a more complicated design such as a confocal microscope, which uses optical sectioning to get better resolution of the fluorescent image.

In some embodiments, confocal microscopy is used for detection and imaging of the detection probe. Confocal microscopy uses point illumination and a pinhole in an optically conjugate plane in front of the detector to eliminate out-of-focus signal. As only light produced by fluorescence very close to the focal plane can be detected, the image's optical resolution, particularly in the sample depth direction, is much better than that of wide-field microscopes. However, as much of the light from sample fluorescence is blocked at the pinhole, this increased resolution is at the cost of decreased signal intensity—so long exposures are often required. As only one point in the sample is illuminated at a time, 2D or 3D imaging requires scanning over a regular raster (e.g., a rectangular pattern of parallel scanning lines) in the specimen. The achievable thickness of the focal plane is defined mostly by the wavelength of the used light divided by the numerical aperture of the objective lens, but also by the optical properties of the specimen. The thin optical sectioning possible makes these types of microscopes particularly good at 3D imaging and surface profiling of samples. CLARITY™-optimized light sheet microscopy (COLM) provides an alternative microscopy for fast 3D imaging of large clarified samples. COLM interrogates large immunostained tissues, permits increased speed of acquisition and results in a higher quality of generated data.

Other types of microscopy that can be employed comprise bright field microscopy, oblique illumination microscopy, dark field microscopy, phase contrast, differential interference contrast (DIC) microscopy, interference reflection microscopy (also known as reflected interference contrast, or RIC), single plane illumination microscopy (SPIM), super-resolution microscopy, laser microscopy, electron microscopy (EM), Transmission electron microscopy (TEM), Scanning electron microscopy (SEM), reflection electron microscopy (REM), Scanning transmission electron microscopy (STEM) and low-voltage electron microscopy (LVEM), scanning probe microscopy (SPM), atomic force microscopy (ATM), ballistic electron emission microscopy (BEEM), chemical force microscopy (CFM), conductive atomic force microscopy (C-AFM), electrochemical scanning tunneling microscope (ECSTM), electrostatic force microscopy (EFM), fluidic force microscope (FluidFM), force modulation microscopy (FMM), feature-oriented scanning probe microscopy (FOSPM), kelvin probe force microscopy (KPFM), magnetic force microscopy (MFM), magnetic resonance force microscopy (MRFM), near-field scanning optical microscopy (NSOM) (or SNOM, scanning near-field optical microscopy, SNOM, Piezoresponse Force Microscopy (PFM), PSTM, photon scanning tunneling microscopy (PSTM), PTMS, photothermal microspectroscopy/microscopy (PTMS), SCM, scanning capacitance microscopy (SCM), SECM, scanning electrochemical microscopy (SECM), SGM, scanning gate microscopy (SGM), SHPM, scanning Hall probe microscopy (SHPM), SICM, scanning ion-conductance microscopy (SICM), SPSM spin polarized scanning tunneling microscopy (SPSM), SSRM, scanning spreading resistance microscopy (SSRM), SThM, scanning thermal microscopy (SThM), STM, scanning tunneling microscopy (STM), STP, scanning tunneling potentiometry (STP), SVM, scanning voltage microscopy (SVM), and synchrotron x-ray scanning tunneling microscopy (SXSTM), and intact tissue expansion microscopy (exM).

In some embodiments, the probe complex is bound to a target sequence in a target nucleic acid, wherein the target sequence is a barcode sequence. In some embodiments, sequential formation of probe complexes comprising amplifier probes and reporter probes bound to the target sequence (e.g., barcode sequence) is used to detect the barcode sequence. In some embodiments, the method comprises forming a first complex comprising a first amplifier probe bound directly or indirectly to the target sequence and to first reporter probes, detecting a signal from the first complex, removing the first amplifier probe and first reporter probes, and forming a second complex comprising a second amplifier bound directly or indirectly to the target sequence and to second reporter probes. In some embodiments, the first signal is a first signal code of a signal code sequence (a temporal “barcode” formed from a temporally sequential series of signals, such as a series of fluorescent colors) that can be used to identify the target nucleic acid molecule (e.g., by identifying the target sequence such as a nucleotide barcode sequence in the target nucleic acid molecule). In some embodiments, the signal code sequence identifies an analyte associated with the target nucleic acid molecule (e.g., a nucleic acid analyte or a non-nucleic acid analyte). The biological sample can be sequentially contacted with pools of amplifier probes and reporter probes (and optionally adapter probes and/or primary probes) any number of times to detect sufficient signals to identify the target sequence.

In some aspects, the analysis and/or sequence determination can be carried out at room temperature for best preservation of tissue morphology with low background noise and error reduction. In some embodiments, the analysis and/or sequence determination comprises eliminating error accumulation as sequencing proceeds. In some embodiments, the analysis and/or sequence determination involves washing to remove unbound polynucleotides, thereafter revealing a fluorescent product for imaging.

### C. Target Nucleic Acid Molecules and Analytes

The methods and compositions disclosed herein can be used to detect and analyze a wide variety of different analytes. In some aspects, an analyte can include any biological substance, structure, moiety, or component to be analyzed. In some aspects, a target disclosed herein may similarly include any analyte of interest. In some examples, a target or analyte is directly or indirectly detected using any of the probe complexes disclosed herein, wherein a probe complex is formed between reporter probes and an amplifier probe comprising at least two reporter hybridization regions separated by a spacer region as described herein. the target nucleic acid is a cellular nucleic acid analyte or a product thereof.

In some embodiments, the target nucleic acid molecule according to the methods disclosed herein is a nucleic acid analyte or is associated with an analyte in the biological sample. For example, a target nucleic acid molecule associated with a nucleic acid analyte can be a product of the analyte such as a cDNA produced from an RNA analyte in the biological sample. In some embodiments, the target nucleic acid molecule is a primary probe hybridized to an analyte or labeling agent in the biological sample, or is a product of a probe bound directly or indirectly to an analyte or labeling agent in the biological sample. In some embodiments, the target nucleic acid molecule is a rolling circle amplification product of a circular probe or circularizable probe or probe set bound directly or indirectly to an analyte or labeling agent in the biological sample. In some embodiments, the target nucleic acid molecule is associated with a non-nucleic acid analyte. In some embodiments, the target nucleic acid is an oligonucleotide reporter in a labeling agent (e.g., as described in Section II.C. (ii)) that binds to an analyte (e.g., an endogenous analyte).

In some embodiments, an analyte herein is endogenous to a biological sample and can include nucleic acid analytes and non-nucleic acid analytes. Methods and compositions disclosed herein can be used to analyze nucleic acid analytes (e.g., using a nucleic acid probe or probe set that directly or indirectly hybridizes to a nucleic acid analyte) and/or non-nucleic acid analytes (e.g., using a labeling agent that comprises a reporter oligonucleotide and binds directly or indirectly to a non-nucleic acid analyte) in any suitable combination.

Analytes can be derived from a specific type of cell and/or a specific sub-cellular region. For example, analytes can be derived from cytosol, from cell nuclei, from mitochondria, from microsomes, and more generally, from any other compartment, organelle, or portion of a cell. Permeabilizing agents that specifically target certain cell compartments and organelles can be used to selectively release analytes from cells for analysis, and/or allow access of one or more reagents (e.g., probes for analyte detection) to the analytes in the cell or cell compartment or organelle.

The analyte may include any biomolecule or chemical compound, including a macromolecule such as a protein or peptide, a lipid or a nucleic acid molecule, or a small molecule, including organic or inorganic molecules. The analyte may be a cell or a microorganism, including a virus, or a fragment or product thereof. An analyte can be any substance or entity for which a specific binding partner (e.g. an affinity binding partner) can be developed. Such a specific binding partner may be a nucleic acid probe (for a nucleic acid analyte) and may lead directly to the generation of a RCA template (e.g. a padlock or other circularizable probe). Alternatively, the specific binding partner may be coupled to a nucleic acid, which may be detected using an RCA strategy, e.g. in an assay which uses or generates a circular nucleic acid molecule which can be the RCA template.

Analytes of particular interest may include nucleic acid molecules, such as DNA (e.g. genomic DNA, mitochondrial DNA, plastid DNA, viral DNA, etc.) and RNA (e.g. mRNA, microRNA, rRNA, snRNA, viral RNA, etc.), and synthetic and/or modified nucleic acid molecules, (e.g. including nucleic acid domains comprising or consisting of synthetic or modified nucleotides such as LNA, PNA, morpholino, etc.), proteinaceous molecules such as peptides, polypeptides, proteins or prions or any molecule which includes a protein or polypeptide component, etc., or fragments thereof, or a lipid or carbohydrate molecule, or any molecule which comprise a lipid or carbohydrate component. The analyte may be a single molecule or a complex that contains two or more molecular subunits, e.g. including but not limited to protein-DNA complexes, which may or may not be covalently bound to one another, and which may be the same or different. Thus in addition to cells or microorganisms, such a complex analyte may also be a protein complex or protein interaction. Such a complex or interaction may thus be a homo- or hetero-multimer. Aggregates of molecules, e.g. proteins may also be target analytes, for example aggregates of the same protein or different proteins. The analyte may also be a complex between proteins or peptides and nucleic acid molecules such as DNA or RNA, e.g. interactions between proteins and nucleic acids, e.g. regulatory factors, such as transcription factors, and DNA or RNA.

### (i) Endogenous Analytes

Examples of non-nucleic acid analytes include, but are not limited to, lipids, carbohydrates, peptides, proteins, glycoproteins (N-linked or O-linked), lipoproteins, phosphoproteins, specific phosphorylated or acetylated variants of proteins, amidation variants of proteins, hydroxylation variants of proteins, methylation variants of proteins, ubiquitylation variants of proteins, sulfation variants of proteins, viral coat proteins, extracellular and intracellular proteins, antibodies, and antigen binding fragments. In some embodiments, the analyte is inside a cell or on a cell surface, such as a transmembrane analyte or one that is attached to the cell membrane. In some embodiments, the analyte can be an organelle (e.g., nuclei or mitochondria). In some embodiments, the analyte is an extracellular analyte, such as a secreted analyte. Exemplary analytes include, but are not limited to, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, an extracellular matrix protein, a posttranslational modification (e.g., phosphorylation, glycosylation, ubiquitination, nitrosylation, methylation, acetylation or lipidation) state of a cell surface protein, a gap junction, and an adherens junction.

Examples of nucleic acid analytes include DNA analytes such as single-stranded DNA (ssDNA), double-stranded DNA (dsDNA), genomic DNA, methylated DNA, specific methylated DNA sequences, fragmented DNA, mitochondrial DNA, in situ synthesized PCR products, and RNA/DNA hybrids. The DNA analyte can be a transcript of another nucleic acid molecule (e.g., DNA or RNA such as mRNA) present in a tissue sample.

Examples of nucleic acid analytes also include RNA analytes such as various types of coding and non-coding RNA. Examples of the different types of RNA analytes include messenger RNA (mRNA), including a nascent RNA, a pre-mRNA, a primary-transcript RNA, and a processed RNA, such as a capped mRNA (e.g., with a 5′ 7-methyl guanosine cap), a polyadenylated mRNA (poly-A tail at the 3′ end), and a spliced mRNA in which one or more introns have been removed. Also included in the analytes disclosed herein are non-capped mRNA, a non-polyadenylated mRNA, and a non-spliced mRNA. The RNA analyte can be a transcript of another nucleic acid molecule (e.g., DNA or RNA such as viral RNA) present in a tissue sample. Examples of a non-coding RNAs (ncRNA) that is not translated into a protein include transfer RNAs (tRNAs) and ribosomal RNAs (rRNAs), as well as small non-coding RNAs such as microRNA (miRNA), small interfering RNA (siRNA), Piwi-interacting RNA (piRNA), small nucleolar RNA (snoRNA), small nuclear RNA (snRNA), extracellular RNA (exRNA), small Cajal body-specific RNAs (scaRNAs), and the long ncRNAs such as Xist and HOTAIR. The RNA can be small (e.g., less than 200 nucleic acid bases in length) or large (e.g., RNA greater than 200 nucleic acid bases in length). Examples of small RNAs include 5.8S ribosomal RNA (rRNA), 5S rRNA, tRNA, miRNA, siRNA, snoRNAs, piRNA, tRNA-derived small RNA (tsRNA), and small rDNA-derived RNA (srRNA). The RNA can be double-stranded RNA or single-stranded RNA. The RNA can be circular RNA. The RNA can be a bacterial rRNA (e.g., 16s rRNA or 23s rRNA).

In some embodiments described herein, an analyte may be a denatured nucleic acid, wherein the resulting denatured nucleic acid is single-stranded. The nucleic acid may be denatured, for example, optionally using formamide, heat, or both formamide and heat. In some embodiments, the nucleic acid is not denatured for use in a method disclosed herein.

Methods and compositions disclosed herein can be used to analyze any number of analytes. For example, the number of analytes that are analyzed can be at least about 2, at least about 3, at least about 4, at least about 5, at least about 6, at least about 7, at least about 8, at least about 9, at least about 10, at least about 11, at least about 12, at least about 13, at least about 14, at least about 15, at least about 20, at least about 25, at least about 30, at least about 40, at least about 50, at least about 100, at least about 1,000, at least about 10,000, at least about 100,000 or more different analytes present in a region of the sample or within an individual feature of the substrate.

### (ii) Labeling Agents

In some embodiments, provided herein are methods and compositions for analyzing endogenous analytes (e.g., RNA, ssDNA, cell surface or intracellular proteins, and/or metabolites) in a sample using one or more labeling agents. In some embodiments, the plurality of probes bind to a target nucleic acid molecule in or associated with a labeling agent, so that the probe complex comprising the amplifier probe and reporter probes is associated with the labeling agent upon hybridization to one or more of the probes. In some embodiments, a labeling agent conjugated to one or more reporter oligonucleotides can be detected using detecting complexes formed between the amplifier probes and the reporter probes, so that the probe complex comprising the amplifier probe and reporter probes is associated with the labeling agent upon hybridization to one or more of the probes. For example, an amplifier probe binds to an amplifier probe hybridization region in a reporter oligonucleotide associated with a labeling agent. In some examples, the reporter oligonucleotide two or more amplifier probe hybridization regions. In some cases, each of the amplifier probes comprises a recognition sequence and at least two reporter hybridization regions as described in Section II.A.

In some embodiments, the target nucleic acid molecule is a rolling circle amplification product associated with the labeling agent. In some embodiments, the rolling circle amplification product is produced from a circular or circularizable probe or probe set that hybridizes to a reporter oligonucleotide in the labeling agent. In some embodiments, the method comprises circularizing the circularizable probe or probe set (e.g., by ligation). The circular or circularized probe can then be amplified by rolling circle amplification. In some embodiments, the rolling circle amplification is performed using a primer, or using the reporter oligonucleotide of the labeling agent as a primer. In some instances, where the reporter oligonucleotide of the labeling agent is used as a primer for rolling circle amplification, the rolling circle amplification product is attached to the labeling agent (via the reporter oligonucleotide incorporated into the RCA product). In some embodiments, the RCA product comprises multiple copies of a barcode sequence corresponding to the labeling agent, allowing for identification of an analyte bound by the labeling agent by analysis of the barcode sequence.

In some embodiments, an analyte labeling agent may include an agent that interacts with an analyte (e.g., an endogenous analyte in a sample). In some embodiments, the labeling agents can comprise a reporter oligonucleotide that is indicative of the analyte or portion thereof interacting with the labeling agent. For example, the reporter oligonucleotide may comprise a barcode sequence that permits identification of the labeling agent. In some cases, the sample contacted by the labeling agent can be further contacted with a probe (e.g., a probe as described in Section II.A comprising a single-stranded probe sequence), that hybridizes to a reporter oligonucleotide of the labeling agent, in order to identify the analyte associated with the labeling agent. In some embodiments, the analyte labeling agent comprises an analyte binding moiety and a labeling agent barcode domain comprising one or more barcode sequences, e.g., a barcode sequence that corresponds to the analyte binding moiety and/or the analyte. In some embodiments, the labeling agent barcode domain comprises at least two copies of a barcode sequence. An analyte binding moiety barcode includes to a barcode that is associated with or otherwise identifies the analyte binding moiety. In some embodiments, by identifying an analyte binding moiety by identifying its associated analyte binding moiety barcode, the analyte to which the analyte binding moiety binds can also be identified. An analyte binding moiety barcode can be a nucleic acid sequence of a given length and/or sequence that is associated with the analyte binding moiety. An analyte binding moiety barcode can generally include any of the variety of aspects of barcodes described herein.

In some embodiments, an amplifier probe binds to an amplifier probe hybridization region in a reporter oligonucleotide associated with a labeling agent. In some embodiments, the amplifier probe comprises a recognition sequence corresponding to a barcode sequence associated with the labeling agent. In some embodiments, the amplifier probe comprises at least two copies of a recognition sequence. In some examples, the reporter oligonucleotide comprises three or more amplifier probe hybridization regions. In some embodiments, the amplifier probe is bound to a reporter oligonucleotide associated with a labeling agent and the amplifier probe binds to at least two reporter probes (e.g., each reporter probe comprises a detectable label). In some embodiments, the amplifier probe is bound to a reporter oligonucleotide associated with a labeling agent and the amplifier probe binds to at least three reporter probes. In some embodiments, the amplifier probe binds directly to a reporter oligonucleotide associated with a labeling agent and the amplifier probe binds to at least three reporter probes.

In some embodiments, provided herein is a method for analyzing a biological sample, comprising contacting the biological sample with an amplifier probe comprising a recognition sequence and at least two reporter hybridization regions, wherein the at least two reporter hybridization regions are separated by a spacer region of between 4 and 8 nucleotides in length, and a plurality of reporter probes, wherein each reporter probe of at least a subset of the reporter probes comprises a detectable label and a reporter region capable of hybridizing to the reporter hybridization region; and detecting a signal from a complex formed by hybridization of at least two reporter probes to the at least two reporter hybridization regions. In some embodiments, the recognition sequence is complementary to a target sequence in a target nucleic acid and the target nucleic acid is an oligonucleotide reporter in a labeling agent that binds to a protein analyte in the biological sample. In some embodiments, the oligonucleotide reporter is conjugated to an antibody that binds to the protein analyte. In some embodiments, the amplifier probe directly binds (e.g., hybridizes) to the oligonucleotide reporter. In some embodiments, each reporter probe directly binds (e.g., hybridizes) to the reporter hybridization regions of the amplifier probe bound to the oligonucleotide reporter conjugated to a labelling agent (e.g., antibody).

In some embodiments, the method comprises one or more post-fixing (also referred to as post-fixation) steps after contacting the sample with one or more labeling agents.

In the methods and systems described herein, one or more labeling agents capable of binding to or otherwise coupling to one or more features may be used to characterize analytes, cells and/or cell features. In some instances, cell features include cell surface features. Analytes may include, but are not limited to, a protein, a receptor, an antigen, a surface protein, a transmembrane protein, a cluster of differentiation protein, a protein channel, a protein pump, a carrier protein, a phospholipid, a glycoprotein, a glycolipid, a cell-cell interaction protein complex, an antigen-presenting complex, a major histocompatibility complex, an engineered T-cell receptor, a T-cell receptor, a B-cell receptor, a chimeric antigen receptor, a gap junction, an adherens junction, or any combination thereof. In some instances, cell features may include intracellular analytes, such as proteins, protein modifications (e.g., phosphorylation status or other post-translational modifications), nuclear proteins, nuclear membrane proteins, or any combination thereof.

In some embodiments, an analyte binding moiety may include any molecule or moiety capable of binding to an analyte (e.g., a biological analyte, e.g., a macromolecular constituent). A labeling agent may include, but is not limited to, a protein, a peptide, an antibody (or an epitope binding fragment thereof), a lipophilic moiety (such as cholesterol), a cell surface receptor binding molecule, a receptor ligand, a small molecule, a bi-specific antibody, a bi-specific T-cell engager, a T-cell receptor engager, a B-cell receptor engager, a pro-body, an aptamer, a monobody, an affimer, a darpin, and a protein scaffold, or any combination thereof. The labeling agents can include (e.g., are attached to) a reporter oligonucleotide that is indicative of the cell surface feature to which the binding group binds. For example, the reporter oligonucleotide may comprise a barcode sequence that permits identification of the labeling agent. For example, a labeling agent that is specific to one type of cell feature (e.g., a first cell surface feature) may have coupled thereto a first reporter oligonucleotide, while a labeling agent that is specific to a different cell feature (e.g., a second cell surface feature) may have a different reporter oligonucleotide coupled thereto. For a description of exemplary labeling agents, reporter oligonucleotides, and methods of use, see, e.g., U.S. Pat. No. 10,550,429; U.S. Pat. Pub. 20190177800; and U.S. Pat. Pub. 20190367969, which are each incorporated by reference herein in their entirety.

In some embodiments, an analyte binding moiety includes one or more antibodies or epitope-binding fragments thereof. The antibodies or epitope-binding fragments including the analyte binding moiety can specifically bind to a target analyte. In some embodiments, the analyte is a protein (e.g., a protein on a surface of the biological sample (e.g., a cell) or an intracellular protein). In some embodiments, a plurality of analyte labeling agents comprising a plurality of analyte binding moieties bind a plurality of analytes present in a biological sample. In some embodiments, the plurality of analytes includes a single species of analyte (e.g., a single species of polypeptide). In some embodiments in which the plurality of analytes includes a single species of analyte, the analyte binding moieties of the plurality of analyte labeling agents are the same. In some embodiments in which the plurality of analytes includes a single species of analyte, the analyte binding moieties of the plurality of analyte labeling agents are the different (e.g., members of the plurality of analyte labeling agents can have two or more species of analyte binding moieties, wherein each of the two or more species of analyte binding moieties binds a single species of analyte, e.g., at different binding sites). In some embodiments, the plurality of analytes includes multiple different species of analyte (e.g., multiple different species of polypeptides).

In other instances, e.g., to facilitate sample multiplexing, a labeling agent that is specific to a particular cell feature may have a first plurality of the labeling agent (e.g., an antibody or lipophilic moiety) coupled to a first reporter oligonucleotide and a second plurality of the labeling agent coupled to a second reporter oligonucleotide.

In some aspects, these reporter oligonucleotides may comprise nucleic acid barcode sequences that permit identification of the labeling agent which the reporter oligonucleotide is coupled to. The selection of oligonucleotides as the reporter may provide advantages of being able to generate significant diversity in terms of sequence, while also being readily attachable to most biomolecules, e.g., antibodies, etc., as well as being readily detected, e.g., using the in situ detection techniques described herein.

Attachment (coupling) of the reporter oligonucleotides to the labeling agents may be achieved through any of a variety of direct or indirect, covalent or non-covalent associations or attachments. For example, oligonucleotides may be covalently attached to a portion of a labeling agent (such a protein, e.g., an antibody or antibody fragment) using chemical conjugation techniques (e.g., Lightning-Link® antibody labeling kits available from Innova Biosciences), as well as other non-covalent attachment mechanisms, e.g., using biotinylated antibodies and oligonucleotides (or beads that include one or more biotinylated linker, coupled to oligonucleotides) with an avidin or streptavidin linker. Antibody and oligonucleotide biotinylation techniques are available. See, e.g., Fang, et al., “Fluoride-Cleavable Biotinylation Phosphoramidite for 5′-end-Labelling and Affinity Purification of Synthetic Oligonucleotides,” Nucleic Acids Res. Jan. 15, 2003; 31(2):708-715, which is entirely incorporated herein by reference for all purposes. Likewise, protein and peptide biotinylation techniques have been developed and are readily available. See, e.g., U.S. Pat. No. 6,265,552, which is entirely incorporated herein by reference for all purposes. Furthermore, click reaction chemistry may be used to couple reporter oligonucleotides to labeling agents. Commercially available kits, such as those from Thunderlink and Abcam, and techniques common in the art may be used to couple reporter oligonucleotides to labeling agents as appropriate. In another example, a labeling agent is indirectly (e.g., via hybridization) coupled to a reporter oligonucleotide comprising a barcode sequence that identifies the label agent. For instance, the labeling agent may be directly coupled (e.g., covalently bound) to a hybridization oligonucleotide that comprises a sequence that hybridizes with a sequence of the reporter oligonucleotide. Hybridization of the hybridization oligonucleotide to the reporter oligonucleotide couples the labeling agent to the reporter oligonucleotide. In some embodiments, the reporter oligonucleotides are releasable from the labeling agent, such as upon application of a stimulus. For example, the reporter oligonucleotide may be attached to the labeling agent through a labile bond (e.g., chemically labile, photolabile, thermally labile, etc.) as generally described for releasing molecules from supports elsewhere herein.

In some cases, the labeling agent can comprise a reporter oligonucleotide and a label. A label can be fluorophore, a radioisotope, a molecule capable of a colorimetric reaction, a magnetic particle, or any other suitable molecule or compound capable of detection. The label can be conjugated to a labeling agent (or reporter oligonucleotide) either directly or indirectly (e.g., the label can be conjugated to a molecule that can bind to the labeling agent or reporter oligonucleotide). In some cases, a label is conjugated to a first oligonucleotide that is complementary (e.g., hybridizes) to a sequence of the reporter oligonucleotide.

In some embodiments, multiple different species of analytes (e.g., polypeptides) from the biological sample can be subsequently associated with the one or more physical properties of the biological sample. For example, the multiple different species of analytes can be associated with locations of the analytes in the biological sample. Such information (e.g., proteomic information when the analyte binding moiety(ies) recognizes a polypeptide(s)) can be used in association with other spatial information (e.g., genetic information from the biological sample, such as DNA sequence information, transcriptome information (e.g., sequences of transcripts), or both). For example, a cell surface protein of a cell can be associated with one or more physical properties of the cell (e.g., a shape, size, activity, or a type of the cell). The one or more physical properties can be characterized by imaging the cell. The cell can be bound by an analyte labeling agent comprising an analyte binding moiety that binds to the cell surface protein and an analyte binding moiety barcode that identifies that analyte binding moiety. Results of protein analysis in a sample (e.g., a tissue sample or a cell) can be associated with DNA and/or RNA analysis in the sample.

(iii) Products of Endogenous Analyte and/or Labeling Agent

In some embodiments, provided herein are methods and compositions for analyzing one or more products of an endogenous analyte and/or a labeling agent in a biological sample. In some embodiments, an endogenous analyte (e.g., a viral or cellular DNA or RNA) or a product (e.g., a hybridization product, a ligation product, an extension product (e.g., by a DNA or RNA polymerase), a replication product, a transcription/reverse transcription product, and/or an amplification product such as a rolling circle amplification (RCA) product) thereof is analyzed. In some embodiments, a labeling agent that directly or indirectly binds to an analyte in the biological sample is analyzed. In some embodiments, a product (e.g., a hybridization product, a ligation product, an extension product (e.g., by a DNA or RNA polymerase), a replication product, a transcription/reverse transcription product, and/or an amplification product such as a rolling circle amplification (RCA) product) of a labeling agent that directly or indirectly binds to an analyte in the biological sample is analyzed.

### (a) Hybridization

In some embodiments, a product of an endogenous analyte and/or a labeling agent is a hybridization product comprising the pairing of substantially complementary or complementary nucleic acid sequences within two different molecules, one of which is the endogenous analyte or the labeling agent (e.g., reporter oligonucleotide attached thereto). The other molecule can be another endogenous molecule or another labeling agent such as a probe (e.g., any described in Section II). In some cases, the hybridization is between an amplifier probe described herein and the target nucleic acid molecule. In some cases, the hybridization is between an adapter probe described herein and the target nucleic acid molecule. In some cases, the hybridization is between a primary probe described herein and the target nucleic acid molecule. Pairing can be achieved by any process in which a nucleic acid sequence joins with a substantially or fully complementary sequence through base pairing to form a hybridization complex. For purposes of hybridization, two nucleic acid sequences are “substantially complementary” if at least 60% (e.g., at least 70%, at least 80%, or at least 90%) of their individual bases are complementary to one another.

Various probes (e.g., primary probes) and probe sets can be hybridized to an endogenous analyte and/or a labeling agent and each probe may comprise one or more barcode sequences. Exemplary barcoded probes or probe sets may be based on a circularizable probe or probe set (e.g., a padlock probe or gapped padlock probe), a SNAIL (Splint Nucleotide Assisted Intramolecular Ligation) probe set, a PLAYR (Proximity Ligation Assay for RNA) probe set, a PLISH (Proximity Ligation in situ Hybridization) probe set, and RNA-templated ligation probes. The specific probe or probe set design can vary. In some embodiments, the target nucleic acid is a rolling circle amplification product produced using any suitable circular or circularizable barcoded probes or probe sets.

### (b) Ligation

In some embodiments, the target nucleic acid molecule analyzed using the amplifier probe and reporter probe complexes herein is a ligation product or an amplification product produced from a ligation product. In some embodiments, the ligation product is formed between two or more endogenous analytes. In some embodiments, the ligation product is formed between two or more labeling agents. In some embodiments, the ligation product is an intramolecular ligation of an endogenous analyte. In some embodiments, the ligation product is an intramolecular ligation product or an intermolecular ligation product, for example, the ligation product can be generated by the circularization of a circularizable probe or probe set upon hybridization to a target sequence. The target sequence can be comprised in an endogenous analyte (e.g., nucleic acid such as a genomic DNA or mRNA) or a product thereof (e.g., cDNA from a cellular mRNA transcript), or in a labeling agent (e.g., the reporter oligonucleotide) or a product thereof.

In some embodiments, the target nucleic acid molecule is a probe or probe set capable of DNA-templated ligation, such as from a cDNA molecule, or an amplification (such as an RCA product) of such a probe or probe set. See, e.g., U.S. Pat. No. 8,551,710, which is hereby incorporated by reference in its entirety. In some embodiments, the target nucleic acid molecule is a probe or probe set capable of RNA-templated ligation, or an amplification (such as an RCA product) of such a probe or probe set. See, e.g., U.S. Pat. Pub. 2020/0224244 which is hereby incorporated by reference in its entirety. In some embodiments, the target nucleic acid molecule is a rolling circle amplification product produced using a SNAIL probe set. See, e.g., U.S. Pat. Pub. 20190055594, which is hereby incorporated by reference in its entirety. In some embodiments, the target nucleic acid molecule is a rolling circle amplification product produced in a multiplexed proximity ligation assay. See, e.g., U.S. Pat. Pub. 20140194311 which is hereby incorporated by reference in its entirety. In some embodiments, the target nucleic acid molecule is a rolling circle amplification product of a probe or probe set capable of proximity ligation, for instance a proximity ligation assay for RNA (e.g., PLAYR) probe set. See, e.g., U.S. Pat. Pub. 20160108458, which is hereby incorporated by reference in its entirety. In some embodiments, the target nucleic acid molecule is a rolling circle amplification product of a circular probe. In some embodiments, a circular probe can be directly or indirectly bound to a nucleic acid analyte or a labeling agent (e.g., a probe for detecting a nucleic acid or non-nucleic acid analyte). In some embodiments, the circular construct is formed from a probe set capable of proximity ligation, for instance a proximity ligation in situ hybridization (PLISH) probe set. See, e.g., U.S. Pat. Pub. 2020/0224243 which is hereby incorporated by reference in its entirety.

In some embodiments, the ligation involves chemical ligation (e.g., click chemistry ligation). In some embodiments, the chemical ligation involves template dependent ligation. In some embodiments, the chemical ligation involves template independent ligation. In some embodiments, the click reaction is a template-independent reaction (see, e.g., Xiong and Seela (2011), J. Org. Chem. 76(14): 5584-5597, incorporated by reference herein in its entirety). In some embodiments, the click reaction is a template-dependent reaction or template-directed reaction. In some embodiments, the template-dependent reaction is sensitive to base pair mismatches such that reaction rate is significantly higher for matched versus unmatched templates. In some embodiments, the click reaction is a nucleophilic addition template-dependent reaction. In some embodiments, the click reaction is a cyclopropane-tetrazine template-dependent reaction.

In some embodiments, the ligation involves enzymatic ligation. In some embodiments, the enzymatic ligation involves use of a ligase. In some aspects, the ligase used herein comprises an enzyme that is commonly used to join polynucleotides together or to join the ends of a single polynucleotide. An RNA ligase, a DNA ligase, or another variety of ligase can be used to ligate two nucleotide sequences together. Ligases comprise ATP-dependent double-strand polynucleotide ligases, NAD-i-dependent double-strand DNA or RNA ligases and single-strand polynucleotide ligases, for example any of the ligases described in EC 6.5.1.1 (ATP-dependent ligases), EC 6.5.1.2 (NAD+-dependent ligases), EC 6.5.1.3 (RNA ligases). Specific examples of ligases comprise bacterial ligases such as E. coli DNA ligase, Tth DNA ligase, Thermococcus sp. (strain 9° N) DNA ligase (9° N™ DNA ligase, New England Biolabs), Taq DNA ligase, Ampligase™ (Epicentre Biotechnologies) and phage ligases such as T3 DNA ligase, T4 DNA ligase and T7 DNA ligase and mutants thereof. In some embodiments, the ligase is a T4 RNA ligase. In some embodiments, the ligase is a splintR ligase. In some embodiments, the ligase is a single stranded DNA ligase. In some embodiments, the ligase is a T4 DNA ligase. In some embodiments, the ligase is a ligase that has an DNA-splinted DNA ligase activity. In some embodiments, the ligase is a ligase that has an RNA-splinted DNA ligase activity.

In some embodiments, the ligation herein is a direct ligation. In some embodiments, the ligation herein is an indirect ligation. “Direct ligation” means that the ends of the polynucleotides hybridize immediately adjacently to one another to form a substrate for a ligase enzyme resulting in their ligation to each other (intramolecular ligation). Alternatively, “indirect” means that the ends of the polynucleotides hybridize non-adjacently to one another, i.e., separated by one or more intervening nucleotides or “gaps”. In some embodiments, said ends are not ligated directly to each other, but instead occurs either via the intermediacy of one or more intervening (so-called “gap” or “gap-filling” (oligo)nucleotides) or by the extension of the 3′ end of a probe to “fill” the “gap” corresponding to said intervening nucleotides (intermolecular ligation). In some cases, the gap of one or more nucleotides between the hybridized ends of the polynucleotides may be “filled” by one or more “gap” (oligo)nucleotide(s) which are complementary to a splint, a circularizable probe or probe set (e.g., a padlock probe), or a target nucleic acid. The gap may be a gap of 1 to 60 nucleotides or a gap of 1 to 40 nucleotides or a gap of 3 to 40 nucleotides. In specific embodiments, the gap may be a gap of about 1, 2, 3, 4, 5, 6, 7, 8, 9 or 10 or more nucleotides, of any integer (or range of integers) of nucleotides in between the indicated values. In some embodiments, the gap between said terminal regions may be filled by a gap oligonucleotide or by extending the 3′ end of a polynucleotide. In some cases, ligation involves ligating the ends of the probe to at least one gap (oligo)nucleotide, such that the gap (oligo)nucleotide becomes incorporated into the resulting polynucleotide. In some embodiments, the ligation herein is preceded by gap filling. In other embodiments, the ligation herein does not require gap filling.

In some embodiments, ligation of the polynucleotides produces polynucleotides with melting temperature higher than that of unligated polynucleotides. Thus, in some aspects, ligation stabilizes the hybridization complex containing the ligated polynucleotides prior to subsequent steps, comprising amplification and detection.

In some aspects, a high fidelity ligase, such as a thermostable DNA ligase (e.g., a Taq DNA ligase), is used. Thermostable DNA ligases are active at elevated temperatures, allowing further discrimination by incubating the ligation at a temperature near the melting temperature (Tₘ) of the DNA strands. This selectively reduces the concentration of annealed mismatched substrates (expected to have a slightly lower Tₘ around the mismatch) over annealed fully base-paired substrates. Thus, high-fidelity ligation can be achieved through a combination of the intrinsic selectivity of the ligase active site and balanced conditions to reduce the incidence of annealed mismatched dsDNA.

In some embodiments, the ligation herein is a proximity ligation of ligating two (or more) nucleic acid sequences that are in proximity with each other, e.g., through enzymatic means (e.g., a ligase). In some embodiments, proximity ligation can include a “gap-filling” step that involves incorporation of one or more nucleic acids by a polymerase, based on the nucleic acid sequence of a template nucleic acid molecule, spanning a distance between the two nucleic acid molecules of interest (see, e.g., U.S. Pat. No. 7,264,929, the entire contents of which are incorporated herein by reference). A wide variety of different methods can be used for proximity ligating nucleic acid molecules, including (but not limited to) “sticky-end” and “blunt-end” ligations. Additionally, single-stranded ligation can be used to perform proximity ligation on a single-stranded nucleic acid molecule. Sticky-end proximity ligations involve the hybridization of complementary single-stranded sequences between the two nucleic acid molecules to be joined, prior to the ligation event itself. Blunt-end proximity ligations generally do not include hybridization of complementary regions from each nucleic acid molecule because both nucleic acid molecules lack a single-stranded overhang at the site of ligation.

### (c) Primer Extension and Amplification

In some embodiments, the target nucleic acid molecule is a primer extension product of an analyte, a labeling agent, a probe or probe set bound to the analyte (e.g., a circularizable probe bound to genomic DNA, mRNA, or cDNA), or a probe or probe set bound to the labeling agent (e.g., a circularizable probe bound to one or more reporter oligonucleotides from the same or different labeling agents).

A primer is generally a single-stranded nucleic acid sequence having a 3′ end that can be used as a substrate for a nucleic acid polymerase in a nucleic acid extension reaction. RNA primers are formed of RNA nucleotides, and are used in RNA synthesis, while DNA primers are formed of DNA nucleotides and used in DNA synthesis. Primers can also include both RNA nucleotides and DNA nucleotides (e.g., in a random or designed pattern). Primers can also include other natural or synthetic nucleotides described herein that can have additional functionality. In some examples, DNA primers can be used to prime RNA synthesis and vice versa (e.g., RNA primers can be used to prime DNA synthesis). Primers can vary in length. For example, primers can be about 6 bases to about 120 bases. For example, primers can include up to about 25 bases. A primer, may in some cases, refer to a primer binding sequence. A primer extension reaction generally refers to any method where two nucleic acid sequences become linked (e.g., hybridized) by an overlap of their respective terminal complementary nucleic acid sequences (e.g., 3′ termini). Such linking can be followed by nucleic acid extension (e.g., an enzymatic extension) of one, or both termini using the other nucleic acid sequence as a template for extension. Enzymatic extension can be performed by an enzyme including, but not limited to, a polymerase and/or a reverse transcriptase.

In some embodiments, the target nucleic acid molecule is a product of an endogenous analyte and/or a labeling agent. In some embodiments, the target nucleic acid molecule is an amplification product of one or more polynucleotides, for instance, a circular probe or circularizable probe or probe set. In some embodiments, the amplifying is achieved by performing rolling circle amplification (RCA). In other embodiments, a primer that hybridizes to the circular probe or circularized probe is added and used as such for amplification. In some embodiments, the RCA comprises a linear RCA, a branched RCA, a dendritic RCA, or any combination thereof.

In some embodiments, the amplification is performed at a temperature between or between about 20° C. and about 60° C. In some embodiments, the amplification is performed at a temperature between or between about 30° C. and about 40° C. In some aspects, the amplification step, such as the rolling circle amplification (RCA) is performed at a temperature between at or about 25° C. and at or about 50° C., such as at or about 25° C., 27° C., 29° C., 31° C., 33° C., 35° C., 37° C., 39° C., 41° C., 43° C., 45° C., 47° C., or 49° C.

In some embodiments, upon addition of a DNA polymerase in the presence of appropriate dNTP precursors and other cofactors, a primer is elongated to produce multiple copies of the circular template. This amplification step can utilize isothermal amplification or non-isothermal amplification. In some embodiments, after the formation of the hybridization complex and association of the amplification probe, the hybridization complex is rolling-circle amplified to generate a cDNA nanoball (i.e., amplicon) containing multiple copies of the cDNA. Techniques for rolling circle amplification (RCA) include linear RCA, a branched RCA, a dendritic RCA, or any combination thereof. (See, e.g., Baner et al, Nucleic Acids Research, 26:5073-5078, 1998; Lizardi et al, Nature Genetics 19:226, 1998; Mohsen et al., Acc Chem Res. 2016 November 15; 49(11): 2540-2550; Schweitzer et al. Proc. Natl Acad. Sci. USA 97:101 13-1 19, 2000; Faruqi et al, BMC Genomics 2:4, 2000; Nallur et al, Nucl. Acids Res. 29:e118, 2001; Dean et al. Genome Res. 1 1:1095-1099, 2001; Schweitzer et al, Nature Biotech. 20:359-365, 2002; U.S. Pat. Nos. 6,054,274, 6,291,187, 6,323,009, 6,344,329 and 6,368,801). Exemplary polymerases for use in RCA comprise DNA polymerase such phi29 (φ29) polymerase, Klenow fragment, Bacillus stearothermophilus DNA polymerase (BST), T4 DNA polymerase, T7 DNA polymerase, or DNA polymerase I. In some aspects, DNA polymerases that have been engineered or mutated to have desirable characteristics can be employed. In some embodiments, the polymerase is phi29 DNA polymerase.

In some aspects, during the amplification step, modified nucleotides can be added to the reaction to incorporate the modified nucleotides in the amplification product (e.g., nanoball). Exemplary of the modified nucleotides comprise amine-modified nucleotides. In some aspects of the methods, for example, for anchoring or cross-linking of the generated amplification product (e.g., nanoball) to a scaffold, to cellular structures and/or to other amplification products (e.g., other nanoballs). In some aspects, the amplification products comprises a modified nucleotide, such as an amine-modified nucleotide. In some embodiments, the amine-modified nucleotide comprises an acrylic acid N-hydroxysuccinimide moiety modification. Examples of other amine-modified nucleotides comprise, but are not limited to, a 5-Aminoallyl-dUTP moiety modification, a 5-Propargylamino-dCTP moiety modification, a N6-6-Aminohexyl-dATP moiety modification, or a 7-Deaza-7-Propargylamino-dATP moiety modification.

In some aspects, the polynucleotides and/or amplification product (e.g., amplicon) can be anchored to a polymer matrix. For example, the polymer matrix can be a hydrogel. In some embodiments, one or more of the polynucleotide probe(s) can be modified to contain functional groups that can be used as an anchoring site to attach the polynucleotide probes and/or amplification product to a polymer matrix. Exemplary modification and polymer matrix that can be employed in accordance with the provided embodiments comprise those described in, for example, WO 2014/163886, WO 2017/079406, U.S. Pat. Nos. 10,494,662, 10,266,888, US 2016/0024555, US 2018/0251833 and US 2017/0219465, which are herein incorporated by reference in their entireties. In some examples, the scaffold also contains modifications or functional groups that can react with or incorporate the modifications or functional groups of the probe set or amplification product. In some examples, the scaffold can comprise oligonucleotides, polymers or chemical groups, to provide a matrix and/or support structures.

The amplification products may be immobilized within the matrix generally at the location of the nucleic acid being amplified, thereby creating a localized colony of amplicons. The amplification products may be immobilized within the matrix by steric factors. The amplification products may also be immobilized within the matrix by covalent or noncovalent bonding. In this manner, the amplification products may be considered to be attached to the matrix. By being immobilized to the matrix, such as by covalent bonding or cross-linking, the size and spatial relationship of the original amplicons is maintained. By being immobilized to the matrix, such as by covalent bonding or cross-linking, the amplification products are resistant to movement or unraveling under mechanical stress.

In some aspects, the amplification products are copolymerized and/or covalently attached to the surrounding matrix thereby preserving their spatial relationship and any information inherent thereto. For example, if the amplification products are those generated from DNA or RNA within a cell embedded in the matrix, the amplification products can also be functionalized to form covalent attachment to the matrix preserving their spatial information within the cell thereby providing a subcellular localization distribution pattern. In some embodiments, the provided methods involve embedding the one or more polynucleotide probe sets and/or the amplification products in the presence of hydrogel subunits to form one or more hydrogel-embedded amplification products. In some embodiments, the hydrogel-tissue chemistry described comprises covalently attaching nucleic acids to in situ synthesized hydrogel for tissue clearing, enzyme diffusion, and multiple-cycle sequencing while an existing hydrogel-tissue chemistry method cannot. In some embodiments, to enable amplification product embedding in the tissue-hydrogel setting, amine-modified nucleotides are comprised in the amplification step (e.g., RCA), functionalized with an acrylamide moiety using acrylic acid N-hydroxysuccinimide esters, and copolymerized with acrylamide monomers to form a hydrogel.

In some embodiments, the RCA template may comprise the target analyte, or a part thereof, where the target analyte is a nucleic acid, or it may be provided or generated as a proxy, or a marker, for the analyte. In some embodiments, different analytes are detected in situ in one or more cells using a RCA-based detection system, e.g., where the signal is provided by generating an RCA product from a circular RCA template which is provided or generated in the assay, and the RCA product is detected to detect the corresponding analyte. The RCA product may thus be regarded as a reporter which is detected to detect the target analyte. However, the RCA template may also be regarded as a reporter for the target analyte; the RCA product is generated based on the RCA template, and comprises complementary copies of the RCA template. The RCA template determines the signal which is detected, and is thus indicative of the target analyte. As will be described in more detail below, the RCA template may be a probe, or a part or component of a probe, or may be generated from a probe, or it may be a component of a detection assay (e.g., a reagent in a detection assay), which is used as a reporter for the assay, or a part of a reporter, or signal-generation system. The RCA template used to generate the RCP may thus be a circular (e.g. circularized) reporter nucleic acid molecule, namely from any RCA-based detection assay which uses or generates a circular nucleic acid molecule as a reporter for the assay. Since the RCA template generates the RCP reporter, it may be viewed as part of the reporter system for the assay.

### III. Compositions and Kits

In some aspects, provided herein are kits for analyzing an analyte in a biological sample according to any of the methods described herein. In some embodiments, provided herein is a kit comprising (i) an amplifier probe comprising a recognition sequence and at least two reporter hybridization regions, wherein the at least two reporter hybridization regions are separated by a spacer region of between 4 and 8 nucleotides in length, and (ii) a plurality of reporter probes, wherein each reporter probe of at least a subset of the reporter probes comprises a detectable label and a reporter region capable of hybridizing to the reporter hybridization region. In some embodiments, the amplifier probe comprises 2, 3, 4, 5, 6, or more reporter hybridization regions. In some embodiments, the amplifier probe comprises between 2 and 20 reporter hybridization regions. In some embodiments, the amplifier probe comprises between 2 and 4, between 2 and 5, or between 2 and 6 reporter hybridization regions.

In some embodiments, the amplifier probe comprises 3 reporter hybridization regions. In some embodiments, the at least two reporter hybridization regions are separated by a spacer region of at least 4 nucleotides in length. In some embodiments, the spacer region is at least 5, at least 6, at least 7, or at least 8 nucleotides in length. In some embodiments, the spacer region is no more than 10, no more than 9, no more than 8, or no more than 7 nucleotides in length. In some embodiments, the spacer region is between 4 and 10, between 4 and 9, between 4 and 8, or between 5 and 8 nucleotides in length. In some embodiments, each of the reporter hybridization regions has the same sequence (e.g., the reporter hybridization regions are copies of the same sequence to allow hybridization of multiple molecules of the same reporter probe to the amplifier probe).

In some embodiments, the spacer region is a sequence of thymidines and/or adenines. In some embodiments, the spacer region is a sequence of thymidines (e.g., 4T (TTTT), 5T (TTTTT), 6T (TTTTTT), 7T (TTTTTTT), or 8T (TTTTTTTT)). In some embodiments, the spacer region is a sequence of adenines (e.g., 4A (AAAA), 5A (AAAAA), 6A (AAAAA), 7A (AAAAAAA), or 8A (AAAAAAAA)). In some embodiment, the spacer region is a sequence of thymidines and adenines (e.g., any sequence of between 4 and 8 nucleotides in length composed on thymidines and adenines). In some embodiments, wherein the amplifier probe comprises multiple spacer regions (e.g., the amplifier probe comprises a first reporter hybridization region, a first spacer, a second reporter hybridization region, a second spacer, and a third reporter hybridization region), each spacer sequence is a sequence of thymidines and/or adenines. The spacer regions can be the same sequence or different sequences.

In some embodiments, provided herein is a kit comprising (i) an amplifier probe comprising a recognition sequence and at least two reporter hybridization regions, wherein the at least two reporter hybridization regions are separated by a spacer region comprising one or more organic spacers, and (ii) a plurality of reporter probes, wherein each reporter probe of at least a subset of the reporter probes comprises a detectable label and a reporter region capable of hybridizing to the reporter hybridization region. In some embodiments, provided herein is a kit comprising (i) an amplifier probe comprising a recognition sequence and at least two reporter hybridization regions, wherein the at least two reporter hybridization regions are separated by a spacer region comprising one or more organic spacers, (ii) a plurality of reporter probes, wherein each reporter probe of at least a subset of the reporter probes comprises a detectable label and a reporter region capable of hybridizing to the reporter hybridization region, and (iii) a labeling agent (e.g., an antibody) conjugated to a oligonucleotide reporter, wherein the recognition sequence of the amplifier probe binds to a sequence of the oligonucleotide reporter. In some embodiments, non-nucleic acid spacers are organic non-nucleic acid spacers. In some embodiments, the spacer region comprises one organic spacer. In some embodiments, the spacer region comprises two or more organic non-nucleic acid spacers (e.g., 2, 3, 4, or more organic non-nucleic acid spacers). Exemplary organic non-nucleic acid spacers are include but are not limited to a triethylene glycol spacer, a C3 spacer phosphoramidite, a hexanediol, an 18-atom hexa-ethyleneglycol spacer, a glycol linker, a carbon chain, and a polyethylene glycol (PEG) spacer. In some embodiments, the spacer region comprises a combination of any of the aforementioned organic non-nucleic acid spacers. In some embodiments, the amplifier probe comprising the organic non-nucleotide spacer is prepared using standard desalting or cartridge purification. In some embodiments, the amplifier probe is purified using HPLC purification.

In some embodiments, the kit further comprises any of the adapter probes and/or primary probes described herein. In some embodiments, the kit further comprises one or more probes or probe sets for generating one or more target nucleic acid molecules associated with analytes in the sample, such as circularizable probes or probe sets for generating rolling circle amplification products associated with nucleic acid or non-nucleic acid analytes in the biological sample (e.g., circularizable probes or probe sets that hybridize to nucleic acid analytes or hybridization, ligation, or extension products thereof in the biological sample, and/or circularizable probes or probe sets that hybridize to oligonucleotide reporters in labeling agents (such as antibodies) that bind to non-nucleic acid analytes in the biological sample). In some embodiments, the kit further comprises any of the labeling agents described herein (e.g., in Section II).

In some embodiments, the kit further comprises one or more labeling agents (e.g., an antibody conjugated to oligonucleotide reporter). In some embodiments, the oligonucleotide reporter comprises an amplifier probe hybridization region. In some embodiments, the oligonucleotide reporter comprises an adapter hybridization region. In some embodiments, the oligonucleotide reporter comprises a target sequence for a primary probe.

The various components of the kit may be present in separate containers or certain compatible components may be pre-combined into a single container. In some embodiments, the kits further contain instructions for using the components of the kit to practice the provided methods.

In some embodiments, the kits can contain reagents and/or consumables required for performing one or more steps of the provided methods. In some embodiments, the kits contain reagents for fixing, embedding, and/or permeabilizing the biological sample. In some embodiments, the kits contain reagents, such as enzymes and buffers for ligation and/or amplification, such as ligases and/or polymerases. In some aspects, the kit can also comprise any of the reagents described herein, e.g., wash buffer and ligation buffer. In some embodiments, the kits contain reagents for detection and/or sequencing, such as barcode detection probes or detectable labels. In some embodiments, the kits optionally contain other components, for example nucleic acid primers, enzymes and reagents, buffers, nucleotides, modified nucleotides, reagents for additional assays.

### IV. Biological Samples and Sample Preparation

A sample disclosed herein can be or derived from any biological sample. Methods and compositions disclosed herein may be used for analyzing a biological sample, which may be obtained from a subject using any of a variety of techniques including, but not limited to, biopsy, surgery, and laser capture microscopy (LCM), and generally includes cells and/or other biological material from the subject. In addition to the subjects described above, a biological sample can be obtained from a prokaryote such as a bacterium, an archaea, a virus, or a viroid. A biological sample can also be obtained from non-mammalian organisms (e.g., a plant, an insect, an arachnid, a nematode, a fungus, or an amphibian). A biological sample can also be obtained from a eukaryote, such as a tissue sample, a patient derived organoid (PDO) or patient derived xenograft (PDX). A biological sample from an organism may comprise one or more other organisms or components therefrom. For example, a mammalian tissue section may comprise a prion, a viroid, a virus, a bacterium, a fungus, or components from other organisms, in addition to mammalian cells and non-cellular tissue components. Subjects from which biological samples can be obtained can be healthy or asymptomatic individuals, individuals that have or are suspected of having a disease (e.g., a patient with a disease such as cancer) or a pre-disposition to a disease, and/or individuals in need of therapy or suspected of needing therapy.

The biological sample can include any number of macromolecules, for example, cellular macromolecules and organelles (e.g., mitochondria and nuclei). The biological sample can include nucleic acids (such as DNA or RNA), proteins/polypeptides, carbohydrates, and/or lipids. In some embodiments, the biological sample is obtained as a tissue sample, such as a tissue section, biopsy, a core biopsy, needle aspirate, or fine needle aspirate. In some embodiments, the biological sample is or comprise a cell pellet or a section of a cell pellet. In some embodiments, the biological sample is or comprise a cell block or a section of a cell block. The sample can be a fluid sample, such as a blood sample, urine sample, or saliva sample. The sample can be a skin sample, a colon sample, a cheek swab, a histology sample, a histopathology sample, a plasma or serum sample, a tumor sample, living cells, cultured cells, a clinical sample such as, for example, whole blood or blood-derived products, blood cells, or cultured tissues or cells, including cell suspensions. In some embodiments, the biological sample may comprise cells which are deposited on a surface.

Biological samples can be derived from a homogeneous culture or population of the subjects or organisms mentioned herein or alternatively from a collection of several different organisms, for example, in a community or ecosystem.

Biological samples can include one or more diseased cells. A diseased cell can have altered metabolic properties, gene expression, protein expression, and/or morphologic features. Examples of diseases include inflammatory disorders, metabolic disorders, nervous system disorders, and cancer. Cancer cells can be derived from solid tumors, hematological malignancies, cell lines, or obtained as circulating tumor cells. Biological samples can also include fetal cells and immune cells.

Biological samples can include analytes (e.g., protein, RNA, and/or DNA) embedded in a 3D matrix. In some embodiments, amplicons (e.g., rolling circle amplification products) derived from or associated with analytes (e.g., protein, RNA, and/or DNA) can be embedded in a 3D matrix. In some embodiments, a 3D matrix may comprise a network of natural molecules and/or synthetic molecules that are chemically and/or enzymatically linked, e.g., by crosslinking. In some embodiments, a 3D matrix may comprise a synthetic polymer. In some embodiments, a 3D matrix comprises a hydrogel.

In some embodiments, a substrate herein is any support that is insoluble in aqueous liquid and which allows for positioning of biological samples, analytes, features, and/or reagents (e.g., probes) on the support. In some embodiments, a biological sample is attached to a substrate. Attachment of the biological sample can be irreversible or reversible, depending upon the nature of the sample and subsequent steps in the analytical method. In certain embodiments, the sample is attached to the substrate reversibly by applying a suitable polymer coating to the substrate, and contacting the sample to the polymer coating. The sample can then be detached from the substrate, e.g., using an organic solvent that at least partially dissolves the polymer coating. Hydrogels are examples of polymers that are suitable for this purpose.

In some embodiments, the substrate can be coated or functionalized with one or more substances to facilitate attachment of the sample to the substrate. Suitable substances that can be used to coat or functionalize the substrate include, but are not limited to, lectins, poly-lysine, antibodies, and polysaccharides.

A variety of steps can be performed to prepare or process a biological sample for and/or during an assay. Except where indicated otherwise, the preparative or processing steps described below can generally be combined in any manner and in any order to appropriately prepare or process a particular sample for and/or analysis.

### (i) Tissue Sectioning

A biological sample can be harvested from a subject (e.g., via surgical biopsy, whole subject sectioning) or grown in vitro on a growth substrate or culture dish as a population of cells, and prepared for analysis as a tissue slice or tissue section. Grown samples may be sufficiently thin for analysis without further processing steps. Alternatively, grown samples, and samples obtained via biopsy or sectioning, can be prepared as thin tissue sections using a mechanical cutting apparatus such as a vibrating blade microtome. As another alternative, in some embodiments, a thin tissue section can be prepared by applying a touch imprint of a biological sample to a suitable substrate material.

The thickness of the tissue section can be a fraction of (e.g., less than 0.9, 0.8, 0.7, 0.6, 0.5, 0.4, 0.3, 0.2, or 0.1) the maximum cross-sectional dimension of a cell. However, tissue sections having a thickness that is larger than the maximum cross-section cell dimension can also be used. For example, cryostat sections can be used, which can be, e.g., 10-20 μm thick.

More generally, the thickness of a tissue section typically depends on the method used to prepare the section and the physical characteristics of the tissue, and therefore sections having a wide variety of different thicknesses can be prepared and used. For example, the thickness of the tissue section can be at least 0.1, 0.2, 0.3, 0.4, 0.5, 0.7, 1.0, 1.5, 2, 3, 4, 5, 6, 7, 8, 9, 10, 12, 13, 14, 15, 20, 30, 40, or 50 μm. Thicker sections can also be used if desired or convenient, e.g., at least 70, 80, 90, or 100 μm or more. Typically, the thickness of a tissue section is between 1-100 μm, 1-50 μm, 1-30 μm, 1-25 μm, 1-20 μm, 1-15 μm, 1-10 μm, 2-8 μm, 3-7 μm, or 4-6 μm, but as mentioned above, sections with thicknesses larger or smaller than these ranges can also be analysed.

Multiple sections can also be obtained from a single biological sample. For example, multiple tissue sections can be obtained from a surgical biopsy sample by performing serial sectioning of the biopsy sample using a sectioning blade. Spatial information among the serial sections can be preserved in this manner, and the sections can be analysed successively to obtain three-dimensional information about the biological sample.

### (ii) Freezing

In some embodiments, the biological sample (e.g., a tissue section as described above) can be prepared by deep freezing at a temperature suitable to maintain or preserve the integrity (e.g., the physical characteristics) of the tissue structure. The frozen tissue sample can be sectioned, e.g., thinly sliced, onto a substrate surface using any number of suitable methods. For example, a tissue sample can be prepared using a chilled microtome (e.g., a cryostat) set at a temperature suitable to maintain both the structural integrity of the tissue sample and the chemical properties of the nucleic acids in the sample. Such a temperature can be, e.g., less than −15° C., less than −20° C., or less than −25° C.

(iii) Fixation and Postfixation

In some embodiments, the biological sample is prepared using formalin-fixation and paraffin-embedding (FFPE), which are established methods. In some embodiments, cell suspensions and other non-tissue samples can be prepared using formalin-fixation and paraffin-embedding. Following fixation of the sample and embedding in a paraffin or resin block, the sample can be sectioned as described above. Prior to analysis, the paraffin-embedding material can be removed from the tissue section (e.g., deparaffinization) by incubating the tissue section in an appropriate solvent (e.g., xylene) followed by a rinse (e.g., 99.5% ethanol for 2 minutes, 96% ethanol for 2 minutes, and 70% ethanol for 2 minutes).

As an alternative to formalin fixation described above, a biological sample can be fixed in any of a variety of other fixatives to preserve the biological structure of the sample prior to analysis. For example, a sample can be fixed via immersion in ethanol, methanol, acetone, paraformaldehyde (PFA)-Triton, and combinations thereof.

In some embodiments, acetone fixation is used with fresh frozen samples, which can include, but are not limited to, cortex tissue, mouse olfactory bulb, human brain tumor, human post-mortem brain, and breast cancer samples. When acetone fixation is performed, pre-permeabilization steps (described below) may not be performed. Alternatively, acetone fixation can be performed in conjunction with permeabilization steps.

In some embodiments, the methods provided herein comprises one or more post-fixing (also referred to as postfixation) steps. In some embodiments, one or more post-fixing step is performed after contacting a sample with a polynucleotide disclosed herein, e.g., one or more probes such as primary probes, amplifier probes, reporter probes, and/or adapter probes described herein. In some embodiments, one or more post-fixing step is performed after a hybridization complex comprising a probe and a target is formed in a sample. In some embodiments, one or more post-fixing step is performed prior to a ligation reaction disclosed herein, such as the ligation of a primary probe (e.g., a circularizable probe or probe set).

In some embodiments, one or more post-fixing step is performed after contacting a sample with a binding or labeling agent (e.g., an antibody or antigen binding fragment thereof) for a non-nucleic acid analyte such as a protein analyte. The labeling agent can comprise a nucleic acid molecule (e.g., reporter oligonucleotide) comprising a sequence corresponding to the labeling agent and therefore corresponds to (e.g., uniquely identifies) the analyte. In some embodiments, the labeling agent can comprise a reporter oligonucleotide comprising one or more barcode sequences.

A post-fixing step may be performed using any suitable fixation reagent disclosed herein, for example, 3% (w/v) paraformaldehyde in DEPC-PBS.

### (iv) Embedding

As an alternative to paraffin embedding described above, a biological sample can be embedded in any of a variety of other embedding materials to provide structural substrate to the sample prior to sectioning and other handling steps. In some cases, the embedding material can be removed e.g., prior to analysis of tissue sections obtained from the sample. Suitable embedding materials include, but are not limited to, waxes, resins (e.g., methacrylate resins), epoxies, and agar.

In some embodiments, the biological sample is embedded in a matrix (e.g., a hydrogel matrix). Embedding the sample in this manner typically involves contacting the biological sample with a hydrogel such that the biological sample becomes surrounded by the hydrogel. For example, the sample can be embedded by contacting the sample with a suitable polymer material, and activating the polymer material to form a hydrogel. In some embodiments, the hydrogel is formed such that the hydrogel is internalized within the biological sample.

In some embodiments, the biological sample is immobilized in the hydrogel via cross-linking of the polymer material that forms the hydrogel. Cross-linking can be performed chemically and/or photochemically, or alternatively by any other suitable hydrogel-formation method.

The composition and application of the hydrogel-matrix to a biological sample typically depends on the nature and preparation of the biological sample (e.g., sectioned, non-sectioned, type of fixation). As one example, where the biological sample is a tissue section, the hydrogel-matrix can include a monomer solution and an ammonium persulfate (APS) initiator/tetramethylethylenediamine (TEMED) accelerator solution. As another example, where the biological sample consists of cells (e.g., cultured cells or cells disassociated from a tissue sample), the cells can be incubated with the monomer solution and APS/TEMED solutions. For cells, hydrogel-matrix gels are formed in compartments, including but not limited to devices used to culture, maintain, or transport the cells. For example, hydrogel-matrices can be formed with monomer solution plus APS/TEMED added to the compartment to a depth ranging from about 0.1 μm to about 2 mm.

Additional methods and aspects of hydrogel embedding of biological samples are described for example in Chen et al., Science 347(6221):543-548, 2015, the entire contents of which are incorporated herein by reference.

### (v) Staining and Immunohistochemistry (IHC)

To facilitate visualization, biological samples can be stained using a wide variety of stains and staining techniques. In some embodiments, for example, a sample can be stained using any number of stains and/or immunohistochemical reagents. One or more staining steps may be performed to prepare or process a biological sample for an assay described herein or may be performed during and/or after an assay. In some embodiments, the sample is contacted with one or more nucleic acid stains, membrane stains (e.g., cellular or nuclear membrane), cytological stains, or combinations thereof. In some examples, the stain may be specific to proteins, phospholipids, DNA (e.g., dsDNA, ssDNA), RNA, an organelle or compartment of the cell. The sample may be contacted with one or more labeled antibodies (e.g., a primary antibody specific for the analyte of interest and a labeled secondary antibody specific for the primary antibody). In some embodiments, cells in the sample can be segmented using one or more images taken of the stained sample.

In some embodiments, the stain is performed using a lipophilic dye. In some examples, the staining is performed with a lipophilic carbocyanine or aminostyryl dye, or analogs thereof (e.g, DiI, DiO, DiR, DiD). Other cell membrane stains may include FM and RH dyes or immunohistochemical reagents specific for cell membrane proteins. In some examples, the stain may include but is not limited to, acridine orange, acid fuchsin, Bismarck brown, carmine, coomassie blue, cresyl violet, DAPI, eosin, ethidium bromide, acid fuchsine, haematoxylin, Hoechst stains, iodine, methyl green, methylene blue, neutral red, Nile blue, Nile red, osmium tetroxide, ruthenium red, propidium iodide, rhodamine (e.g., rhodamine B), or safranine, or derivatives thereof. In some embodiments, the sample may be stained with haematoxylin and eosin (H&E).

The sample can be stained using hematoxylin and eosin (H&E) staining techniques, using Papanicolaou staining techniques, Masson's trichrome staining techniques, silver staining techniques, Sudan staining techniques, and/or using Periodic Acid Schiff (PAS) staining techniques. PAS staining is typically performed after formalin or acetone fixation. In some embodiments, the sample can be stained using Romanowsky stain, including Wright's stain, Jenner's stain, Can-Grunwald stain, Leishman stain, and Giemsa stain.

In some embodiments, biological samples is destained. Any suitable methods of destaining or discoloring a biological sample may be utilized and generally depend on the nature of the stain(s) applied to the sample. For example, in some embodiments, one or more immunofluorescent stains are applied to the sample via antibody coupling. Such stains can be removed using techniques such as cleavage of disulfide linkages via treatment with a reducing agent and detergent washing, chaotropic salt treatment, treatment with antigen retrieval solution, and treatment with an acidic glycine buffer. Methods for multiplexed staining and destaining are described, for example, in Bolognesi et al., J. Histochem. Cytochem. 2017; 65(8): 431-444, Lin et al., Nat Commun. 2015; 6:8390, Pirici et al., J. Histochem. Cytochem. 2009; 57:567-75, and Glass et al., J. Histochem. Cytochem. 2009; 57:899-905, the entire contents of each of which are incorporated herein by reference.

### (vi) Isometric Expansion

In some embodiments, a biological sample embedded in a matrix (e.g., a hydrogel) is isometrically expanded. Isometric expansion methods that can be used include hydration, a preparative step in expansion microscopy, as described in, e.g., Chen et al., Science 347(6221):543-548, 2015 and U.S. Pat. No. 10,059,990, which are herein incorporated by reference in their entireties.

Isometric expansion can be performed by anchoring one or more components of a biological sample to a gel, followed by gel formation, proteolysis, and swelling. In some embodiments, analytes in the sample, products of the analytes, and/or probes associated with analytes in the sample can be anchored to the matrix (e.g., hydrogel). Isometric expansion of the biological sample can occur prior to immobilization of the biological sample on a substrate, or after the biological sample is immobilized to a substrate. In some embodiments, the isometrically expanded biological sample can be removed from the substrate prior to contacting the substrate with probes disclosed herein.

In general, the steps used to perform isometric expansion of the biological sample can depend on the characteristics of the sample (e.g., thickness of tissue section, fixation, cross-linking), and/or the analyte of interest (e.g., different conditions to anchor RNA, DNA, and protein to a gel).

In some embodiments, proteins in the biological sample are anchored to a swellable gel such as a polyelectrolyte gel. An antibody can be directed to the protein before, after, or in conjunction with being anchored to the swellable gel. DNA and/or RNA in a biological sample can also be anchored to the swellable gel via a suitable linker. Examples of such linkers include, but are not limited to, 6-((Acryloyl)amino) hexanoic acid (Acryloyl-X SE) (available from ThermoFisher, Waltham, MA), Label-IT Amine (available from MirusBio, Madison, WI) and Label X (described for example in Chen et al., Nat. Methods 13:679-684, 2016 and U.S. Pat. No. 10,059,990, the entire contents of which are incorporated herein by reference).

Isometric expansion of the sample can increase the spatial resolution of the subsequent analysis of the sample. The increased resolution in spatial profiling can be determined by comparison of an isometrically expanded sample with a sample that has not been isometrically expanded.

In some embodiments, a biological sample is isometrically expanded to a size at least 2×, 2.1×, 2.2×, 2.3×, 2.4×, 2.5×, 2.6×, 2.7×, 2.8×, 2.9×, 3×, 3.1×, 3.2×, 3.3×, 3.4×, 3.5×, 3.6×, 3.7×, 3.8×, 3.9×, 4×, 4.1×, 4.2×, 4.3×, 4.4×, 4.5×, 4.6×, 4.7×, 4.8×, or 4.9× its non-expanded size. In some embodiments, the sample is isometrically expanded to at least 2× and less than 20× of its non-expanded size.

(vii) Crosslinking and De-Crosslinking

In some embodiments, the biological sample is reversibly cross-linked prior to or during an in situ assay. In some aspects, the analytes, polynucleotides and/or amplification product (e.g., amplicon) of an analyte or a probe bound thereto are anchored to a polymer matrix. For example, the polymer matrix can be a hydrogel. In some embodiments, one or more of the polynucleotide probe(s) and/or amplification product (e.g., amplicon) thereof are modified to contain functional groups that can be used as an anchoring site to attach the polynucleotide probes and/or amplification product to a polymer matrix. In some embodiments, a modified probe comprising oligo dT may be used to bind to mRNA molecules of interest, followed by reversible or irreversible crosslinking of the mRNA molecules.

In some embodiments, the biological sample is immobilized in a hydrogel via cross-linking of the polymer material that forms the hydrogel. Cross-linking can be performed chemically and/or photochemically, or alternatively by any other suitable hydrogel-formation method. A hydrogel may include a macromolecular polymer gel including a network. Within the network, some polymer chains can optionally be cross-linked, although cross-linking does not always occur.

In some embodiments, a hydrogel includes hydrogel subunits, such as, but not limited to, acrylamide, bis-acrylamide, polyacrylamide and derivatives thereof, poly(ethylene glycol) and derivatives thereof (e.g. PEG-acrylate (PEG-DA), PEG-RGD), gelatin-methacryloyl (GelMA), methacrylated hyaluronic acid (MeHA), polyaliphatic polyurethanes, polyether polyurethanes, polyester polyurethanes, polyethylene copolymers, polyamides, polyvinyl alcohols, polypropylene glycol, polytetramethylene oxide, polyvinyl pyrrolidone, polyacrylamide, poly(hydroxyethyl acrylate), and poly(hydroxyethyl methacrylate), collagen, hyaluronic acid, chitosan, dextran, agarose, gelatin, alginate, protein polymers, methylcellulose, and the like, and combinations thereof.

In some embodiments, a hydrogel includes a hybrid material, e.g., the hydrogel material includes elements of both synthetic and natural polymers. Examples of suitable hydrogels are described, for example, in U.S. Pat. Nos. 6,391,937, 9,512,422, and 9,889,422, and in U.S. Patent Application Publication Nos. 2017/0253918, 2018/0052081 and 2010/0055733, the entire contents of each of which are incorporated herein by reference.

In some embodiments, the hydrogel can form the substrate. In some embodiments, the substrate includes a hydrogel and one or more second materials. In some embodiments, the hydrogel is placed on top of one or more second materials. For example, the hydrogel can be pre-formed and then placed on top of, underneath, or in any other configuration with one or more second materials. In some embodiments, hydrogel formation occurs after contacting one or more second materials during formation of the substrate. Hydrogel formation can also occur within a structure (e.g., wells, ridges, projections, and/or markings) located on a substrate.

In some embodiments, hydrogel formation on a substrate occurs before, contemporaneously with, or after probes are provided to the sample. For example, hydrogel formation can be performed on the substrate already containing the probes.

In some embodiments, hydrogel formation occurs within a biological sample. In some embodiments, a biological sample (e.g., tissue section) is embedded in a hydrogel. In some embodiments, hydrogel subunits are infused into the biological sample, and polymerization of the hydrogel is initiated by an external or internal stimulus.

In embodiments in which a hydrogel is formed within a biological sample, functionalization chemistry is used. In some embodiments, functionalization chemistry includes hydrogel-tissue chemistry (HTC). Any hydrogel-tissue backbone (e.g., synthetic or native) suitable for HTC can be used for anchoring biological macromolecules and modulating functionalization. Non-limiting examples of methods using HTC backbone variants include CLARITY, PACT, ExM, SWITCH and ePACT. In some embodiments, hydrogel formation within a biological sample is permanent. For example, biological macromolecules can permanently adhere to the hydrogel allowing multiple rounds of interrogation. In some embodiments, hydrogel formation within a biological sample is reversible.

In some embodiments, additional reagents are added to the hydrogel subunits before, contemporaneously with, and/or after polymerization. For example, additional reagents can include but are not limited to oligonucleotides (e.g., probes), endonucleases to fragment DNA, fragmentation buffer for DNA, DNA polymerase enzymes, dNTPs used to amplify the nucleic acid and to attach the barcode to the amplified fragments. Other enzymes can be used, including without limitation, RNA polymerase, ligase, proteinase K, and DNAse. Additional reagents can also include reverse transcriptase enzymes, including enzymes with terminal transferase activity, primers, and switch oligonucleotides. In some embodiments, optical labels are added to the hydrogel subunits before, contemporaneously with, and/or after polymerization.

In some embodiments, HTC reagents are added to the hydrogel before, contemporaneously with, and/or after polymerization. In some embodiments, a cell labeling agent is added to the hydrogel before, contemporaneously with, and/or after polymerization. In some embodiments, a cell-penetrating agent is added to the hydrogel before, contemporaneously with, and/or after polymerization.

Hydrogels embedded within biological samples can be cleared using any suitable method. For example, electrophoretic tissue clearing methods can be used to remove biological macromolecules from the hydrogel-embedded sample. In some embodiments, a hydrogel-embedded sample is stored before or after clearing of hydrogel, in a medium (e.g., a mounting medium, methylcellulose, or other semi-solid mediums).

In some embodiments, a method disclosed herein comprises de-crosslinking the reversibly cross-linked biological sample. The de-crosslinking does not need to be complete. In some embodiments, only a portion of crosslinked molecules in the reversibly cross-linked biological sample are de-crosslinked and allowed to migrate.

(viii) Tissue Permeabilization and Treatment

In some embodiments, a biological sample is permeabilized to facilitate transfer of species (such as probes) into the sample. If a sample is not permeabilized sufficiently, the transfer of species (such as probes) into the sample may be too low to enable adequate analysis. Conversely, if the tissue sample is too permeable, the relative spatial relationship of the analytes within the tissue sample can be lost. Hence, a balance between permeabilizing the tissue sample enough to obtain good signal intensity while still maintaining the spatial resolution of the analyte distribution in the sample is desirable.

In general, a biological sample can be permeabilized by exposing the sample to one or more permeabilizing agents. Suitable agents for this purpose include, but are not limited to, organic solvents (e.g., acetone, ethanol, and methanol), cross-linking agents (e.g., paraformaldehyde), detergents (e.g., saponin, Triton X-100™ or Tween-20™), and enzymes (e.g., trypsin, proteases). In some embodiments, the biological sample can be incubated with a cellular permeabilizing agent to facilitate permeabilization of the sample. Additional methods for sample permeabilization are described, for example, in Jamur et al., Method Mol. Biol. 588:63-66, 2010, the entire contents of which are incorporated herein by reference. Any suitable method for sample permeabilization can generally be used in connection with the samples described herein.

In some embodiments, the biological sample is permeabilized by adding one or more lysis reagents to the sample. Examples of suitable lysis agents include, but are not limited to, bioactive reagents such as lysis enzymes that are used for lysis of different cell types, e.g., gram positive or negative bacteria, plants, yeast, mammalian, such as lysozymes, achromopeptidase, lysostaphin, labiase, kitalase, lyticase, and a variety of other commercially available lysis enzymes.

Other lysis agents can additionally or alternatively be added to the biological sample to facilitate permeabilization. For example, surfactant-based lysis solutions can be used to lyse sample cells. Lysis solutions can include ionic surfactants such as, for example, sarcosyl and sodium dodecyl sulfate (SDS). More generally, chemical lysis agents can include, without limitation, organic solvents, chelating agents, detergents, surfactants, and chaotropic agents.

In some embodiments, the biological sample is permeabilized by non-chemical permeabilization methods. For example, non-chemical permeabilization methods that can be used include, but are not limited to, physical lysis techniques such as electroporation, mechanical permeabilization methods (e.g., bead beating using a homogenizer and grinding balls to mechanically disrupt sample tissue structures), acoustic permeabilization (e.g., sonication), and thermal lysis techniques such as heating to induce thermal permeabilization of the sample.

Additional reagents can be added to a biological sample to perform various functions prior to analysis of the sample. In some embodiments, DNase and RNase inactivating agents or inhibitors such as proteinase K, and/or chelating agents such as EDTA, can be added to the sample. For example, a method disclosed herein may comprise a step for increasing accessibility of a nucleic acid for binding, e.g., a denaturation step to open up DNA in a cell for hybridization by a probe. For example, proteinase K treatment may be used to free up DNA with proteins bound thereto.

### (ix) Selective Enrichment of RNA Species

In some embodiments, where RNA is the analyte, one or more RNA analyte species of interest is selectively enriched. For example, one or more species of RNA of interest can be selected by addition of one or more oligonucleotides to the sample. In some embodiments, the additional oligonucleotide is a sequence used for priming a reaction by an enzyme (e.g., a polymerase). For example, one or more primer sequences with sequence complementarity to one or more RNAs of interest can be used to amplify the one or more RNAs of interest, e.g., to generate cDNA, thereby selectively enriching these RNAs.

In some aspects, when two or more analytes are analyzed, a first and second probe that is specific for (e.g., specifically hybridizes to) each RNA or cDNA analyte are used. For example, in some embodiments of the methods provided herein, templated ligation is used to detect gene expression in a biological sample. An analyte of interest (such as a protein), bound by a labeling agent or binding agent (e.g., an antibody or epitope binding fragment thereof), wherein the binding agent is conjugated or otherwise associated with a reporter oligonucleotide comprising a reporter sequence that identifies the binding agent, can be targeted for analysis. Probes may be hybridized to the reporter oligonucleotide and ligated in a templated ligation reaction to generate a product for analysis. In some embodiments, gaps between the probe oligonucleotides may first be filled prior to ligation, using, for example, Mu polymerase, DNA polymerase, RNA polymerase, reverse transcriptase, VENT polymerase, Taq polymerase, and/or any combinations, derivatives, and variants (e.g., engineered mutants) thereof. In some embodiments, the assay can further include extension or amplification of templated ligation products (e.g., by rolling circle amplification of a circular product generated in a templated ligation reaction).

A biological sample may comprise one or a plurality of analytes of interest. Methods for performing multiplexed assays to analyze two or more different analytes using the sequential formation and removal of the probe complexes disclosed herein in a single biological sample are provided.

### V. Applications

In some aspects, the provided embodiments can be applied in an in situ method of analyzing nucleic acid sequences, such as an in situ transcriptomic analysis or in situ sequencing, for example from intact tissues or samples in which the spatial information has been preserved. In some aspects, the embodiments can be applied in an imaging or detection method for multiplexed nucleic acid analysis.

In some aspects, the embodiments can be applied in investigative and/or diagnostic applications, for example, for characterization or assessment of particular cell or a tissue from a subject. Applications of the provided method can comprise biomedical research and clinical diagnostics. For example, in biomedical research, applications comprise, but are not limited to, spatially resolved gene expression analysis for biological investigation or drug screening. In clinical diagnostics, applications comprise, but are not limited to, detecting gene markers such as disease, immune responses, bacterial or viral DNA/RNA for patient samples.

In some aspects, the embodiments can be applied to visualize the distribution of genetically encoded markers in whole tissue at subcellular resolution, for example, chromosomal abnormalities (inversions, duplications, translocations, etc.), loss of genetic heterozygosity, the presence of gene alleles indicative of a predisposition towards disease or good health, likelihood of responsiveness to therapy, or in personalized medicine or ancestry.

### VI. Opto-Fluidic Instruments for Analysis of Biological Samples

Provided herein is an instrument having integrated optics and fluidics modules (an “opto-fluidic instrument” or “opto-fluidic system”) for detecting target molecules (e.g., nucleic acids, proteins, antibodies, etc.) in biological samples (e.g., one or more cells or a tissue sample) as described herein. In an opto-fluidic instrument, the fluidics module is configured to deliver one or more reagents (e.g., amplifier probe(s), reporter probes, and optionally primary probes and/or adapter probes) to the biological sample and/or remove spent reagents therefrom. Additionally, the optics module is configured to illuminate the biological sample with light having one or more spectral emission curves (over a range of wavelengths) and subsequently capture one or more images of emitted light signals from the biological sample during one or more analytical cycles (e.g., as described in Section II). In various embodiments, the captured images may be processed in real time and/or at a later time to determine the presence of the one or more target molecules in the biological sample, as well as three-dimensional position information associated with each detected target molecule. Additionally, the opto-fluidics instrument includes a sample module configured to receive (and, optionally, secure) one or more biological samples. In some instances, the sample module includes an X-Y stage configured to move the biological sample along an X-Y plane (e.g., perpendicular to an objective lens of the optics module).

In various embodiments, the opto-fluidic instrument is configured to analyze one or more target molecules in their naturally occurring place (i.e., in situ) within the biological sample. For example, an opto-fluidic instrument may be an in-situ analysis system used to analyze a biological sample and detect target molecules including but not limited to DNA, RNA, proteins, antibodies, and/or the like.

It is to be noted that, although the above discussion relates to an opto-fluidic instrument that can be used for in situ target molecule detection using a complex formed by hybridization of at least two reporter probes to at least two reporter hybridization regions in an amplifier probe, the discussion herein equally applies to any opto-fluidic instrument that employs any imaging or target molecule detection technique. That is, for example, an opto-fluidic instrument may include a fluidics module that includes fluids needed for establishing the experimental conditions required for the probing of target molecules in the sample. Further, such an opto-fluidic instrument may also include a sample module configured to receive the sample, and an optics module including an imaging system for illuminating (e.g., exciting one or more fluorescent probes (e.g., reporter probes) within the sample) and/or imaging light signals received from the probed sample. The in-situ analysis system may also include other ancillary modules configured to facilitate the operation of the opto-fluidic instrument, such as, but not limited to, cooling systems, motion calibration systems, etc.

### VII. Terminology

Unless defined otherwise, all terms of art, notations and other technical and scientific terms or terminology used herein are intended to have the same meaning as is commonly understood by one of ordinary skill in the art to which the claimed subject matter pertains. In some cases, terms with commonly understood meanings are defined herein for clarity and/or for ready reference, and the inclusion of such definitions herein should not necessarily be construed to represent a substantial difference over what is generally understood in the art.

The terms “polynucleotide,” “polynucleotide,” and “nucleic acid molecule,” used interchangeably herein, refer to polymeric forms of nucleotides of any length, either ribonucleotides or deoxyribonucleotides. Thus, this term comprises, but is not limited to, single-, double-, or multi-stranded DNA or RNA, genomic DNA, cDNA, DNA-RNA hybrids, or a polymer comprising purine and pyrimidine bases or other natural, chemically or biochemically modified, non-natural, or derivatized nucleotide bases. The backbone of the polynucleotide can comprise sugars and phosphate groups (as may typically be found in RNA or DNA), or modified or substituted sugar or phosphate groups.

“Hybridization” as used herein may refer to the process in which two single-stranded polynucleotides bind non-covalently to form a stable double-stranded polynucleotide. In one aspect, the resulting double-stranded polynucleotide can be a “hybrid” or “duplex.” “Hybridization conditions” typically include salt concentrations of approximately less than 1 M, often less than about 500 mM and may be less than about 200 mM. A “hybridization buffer” includes a buffered salt solution such as 5% SSPE, or other such buffers utilized during nucleic acid hybridization. Hybridization temperatures can be as low as 5° C., but are typically greater than 22° C., and more typically greater than about 30° C., and typically in excess of 37° C. Hybridizations are often performed under stringent conditions, e.g., conditions under which a sequence will hybridize to its target sequence but will not hybridize to other, non-complementary sequences. Stringent conditions are sequence-dependent and are different in different circumstances. For example, longer fragments may require higher hybridization temperatures for specific hybridization than short fragments. As other factors may affect the stringency of hybridization, including base composition and length of the complementary strands, presence of organic solvents, and the extent of base mismatching, the combination of parameters is more important than the absolute measure of any one parameter alone. Generally stringent conditions are selected to be about 5° C. lower than the Tₘ for the specific sequence at a defined ionic strength and pH. The melting temperature Tₘ can be the temperature at which a population of double-stranded nucleic acid molecules becomes half dissociated into single strands. Several equations for calculating the Tₘ of nucleic acids have been established. As indicated by standard references, a simple estimate of the Tₘ value may be calculated by the equation, Tₘ=81.5+0.41 (% G+C), when a nucleic acid is in aqueous solution at 1 M NaCl (see e.g., Anderson and Young, Quantitative Filter Hybridization, in Nucleic Acid Hybridization (1985)). Other references (e.g., Allawi and SantaLucia, Jr., Biochemistry, 36:10581-94 (1997)) include alternative methods of computation which take structural and environmental, as well as sequence characteristics into account for the calculation of Tₘ.

In general, the stability of a hybrid is a function of the ion concentration and temperature. Typically, a hybridization reaction is performed under conditions of lower stringency, followed by washes of varying, but higher, stringency. Exemplary stringent conditions include a salt concentration of at least 0.01 M to no more than 1 M sodium ion concentration (or other salt) at a pH of about 7.0 to about 8.3 and a temperature of at least 25° C. For example, conditions of 5×SSPE (750 mM NaCl, 50 mM sodium phosphate, 5 mM EDTA at pH 7.4) and a temperature of approximately 30° C. are suitable for allele-specific hybridizations, though a suitable temperature depends on the length and/or GC content of the region hybridized. In one aspect, “stringency of hybridization” in determining percentage mismatch can be as follows: 1) high stringency: 0.1×SSPE, 0.1% SDS, 65° C.; 2) medium stringency: 0.2×SSPE, 0.1% SDS, 50° C. (also referred to as moderate stringency); and 3) low stringency: 1.0×SSPE, 0.1% SDS, 50° C. It is understood that equivalent stringencies may be achieved using alternative buffers, salts and temperatures. For example, moderately stringent hybridization can refer to conditions that permit a nucleic acid molecule such as a probe to bind a complementary nucleic acid molecule. The hybridized nucleic acid molecules generally have at least 60% identity, including for example at least any of 70%, 75%, 80%, 85%, 90%, or 95% identity. Moderately stringent conditions can be conditions equivalent to hybridization in 50% formamide, 5×Denhardt's solution, 5×SSPE, 0.2% SDS at 42° C., followed by washing in 0.2×SSPE, 0.2% SDS, at 42° C. High stringency conditions can be provided, for example, by hybridization in 50% formamide, 5×Denhardt's solution, 5×SSPE, 0.2% SDS at 42° C., followed by washing in 0.1×SSPE, and 0.1% SDS at 65° C. Low stringency hybridization can refer to conditions equivalent to hybridization in 10% formamide, 5×Denhardt's solution, 6×SSPE, 0.2% SDS at 22° C., followed by washing in 1×SSPE, 0.2% SDS, at 37° C. Denhardt's solution contains 1% Ficoll, 1% polyvinylpyrolidone, and 1% bovine serum albumin (BSA). 20×SSPE (sodium chloride, sodium phosphate, ethylene diamide tetraacetic acid (EDTA)) contains 3M sodium chloride, 0.2M sodium phosphate, and 0.025 M EDTA. Other suitable moderate stringency and high stringency hybridization buffers and conditions have been described, for example, in Sambrook et al., Molecular Cloning: A Laboratory Manual, 2nd ed., Cold Spring Harbor Press, Plainview, N.Y. (1989); and Ausubel et al., Short Protocols in Molecular Biology, 4th ed., John Wiley & Sons (1999).

Alternatively, substantial complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See M. Kanehisa, Nucleic Acids Res. 12:203 (1984).

A “primer” used herein, in some embodiments, is an oligonucleotide, either natural or synthetic, that is capable, upon forming a duplex with a polynucleotide template, of acting as a point of initiation of nucleic acid synthesis and being extended from its 3′ end along the template so that an extended duplex is formed. The sequence of nucleotides added during the extension process is determined by the sequence of the template polynucleotide. Primers usually are extended by a DNA polymerase.

In some instances, “ligation” refers to the formation of a covalent bond or linkage between the termini of two or more nucleic acids, e.g., oligonucleotides and/or polynucleotides, in a template-driven reaction. The nature of the bond or linkage may vary widely and the ligation may be carried out enzymatically or chemically. As used herein, ligations are usually carried out enzymatically to form a phosphodiester linkage between a 5′ carbon terminal nucleotide of one oligonucleotide with a 3′ carbon of another nucleotide.

“Sequencing,” “sequence determination” and the like means determination of information relating to the nucleotide base sequence of a nucleic acid. Such information may include the identification or determination of partial as well as full sequence information of the nucleic acid. Sequence information may be determined with varying degrees of statistical reliability or confidence. In one aspect, the term includes the determination of the identity and ordering of a plurality of contiguous nucleotides in a nucleic acid. “High throughput digital sequencing” or “next generation sequencing” means sequence determination using methods that determine many (typically thousands to billions) of nucleic acid sequences in an intrinsically parallel manner, i.e., where DNA templates are prepared for sequencing not one at a time, but in a bulk process, and where many sequences are read out preferably in parallel, or alternatively using an ultra-high throughput serial process that itself may be parallelized. Such methods include but are not limited to pyrosequencing (for example, as commercialized by 454 Life Sciences, Inc., Branford, Conn.); sequencing by ligation (for example, as commercialized in the SOLiD™ technology, Life Technologies, Inc., Carlsbad, Calif.); sequencing by synthesis using modified nucleotides (such as commercialized in TruSeq™ and HiSeq™ technology by Illumina, Inc., San Diego, Calif; HeliScope™ by Helicos Biosciences Corporation, Cambridge, Ma.; and PacBio RS by Pacific Biosciences of California, Inc., Menlo Park, Calif.), sequencing by ion detection technologies (such as Ion Torrent™ technology, Life Technologies, Carlsbad, Calif); sequencing of DNA nanoballs (Complete Genomics, Inc., Mountain View, Calif.); nanopore-based sequencing technologies (for example, as developed by Oxford Nanopore Technologies, LTD, Oxford, UK), and like highly parallelized sequencing methods. “Multiplexing” or “multiplex assay” herein may refer to an assay or other analytical method in which the presence and/or amount of multiple targets, e.g., multiple nucleic acid target sequences, can be assayed simultaneously by using more than one capture probe conjugate, each of which has at least one different detection characteristic, e.g., fluorescence characteristic (for example excitation wavelength, emission wavelength, emission intensity, FWHM (full width at half maximum peak height), or fluorescence lifetime) or a unique nucleic acid or protein sequence characteristic.

The term “about” as used herein refers to the usual error range for the respective value readily known to the skilled person in this technical field. Reference to “about” a value or parameter herein comprises (and describes) embodiments that are directed to that value or parameter per se.

As used herein, the singular forms “a,” “an,” and “the” comprise plural referents unless the context clearly dictates otherwise. For example, “a” or “an” means “at least one” or “one or more.”

Throughout this disclosure, various aspects of the claimed subject matter are presented in a range format. It should be understood that the description in range format is merely for convenience and brevity and should not be construed as an inflexible limitation on the scope of the claimed subject matter. Accordingly, the description of a range should be considered to have specifically disclosed all the possible sub-ranges as well as individual numerical values within that range. For example, where a range of values is provided, it is understood that each intervening value, between the upper and lower limit of that range and any other stated or intervening value in that stated range is encompassed within the claimed subject matter. The upper and lower limits of these smaller ranges may independently be comprised in the smaller ranges, and are also encompassed within the claimed subject matter, subject to any specifically excluded limit in the stated range. Where the stated range comprises one or both of the limits, ranges excluding either or both of those comprised limits are also comprised in the claimed subject matter. This applies regardless of the breadth of the range.

Use of ordinal terms such as “first”, “second”, “third”, etc., in the claims to modify a claim element does not by itself connote any priority, precedence, or order of one claim element over another or the temporal order in which acts of a method are performed, but are used merely as labels to distinguish one claim element having a certain name from another element having a same name (but for use of the ordinal term) to distinguish the claim elements. Similarly, use of a), b), etc., or i), ii), etc. does not by itself connote any priority, precedence, or order of steps in the claims. Similarly, the use of these terms in the specification does not by itself connote any required priority, precedence, or order.

### EXAMPLES

The following examples are included for illustrative purposes only and are not intended to limit the scope of the present disclosure.

### Example 1: Spacer Regions Between Reporter Hybridization Regions Improve Signal Intensity

This example provides results demonstrating increased signal intensity using spacers of various lengths between multiple reporter hybridization regions in an amplifier probe, wherein the reporter hybridization regions hybridize to fluorescently labeled reporter probes. Signal intensity was further increased by using longer spacers between multiple amplifier probe hybridization regions in an adapter probe.

Amplifier probes were designed comprising three reporter hybridization regions separated by spacer regions, as in the schematic illustration of an amplifier probe shown in FIG. 1. Three different spacer regions were used to test the effect of spacer regions of various lengths between the reporter hybridization regions: (1) a sequence two nucleotide residues in length (2T's; estimated length of approximately 1.35 nm), (2) an 18-atom hexa-ethyleneglycol spacer (iSp18; estimated length of approximately 2.5 nm), and (3) a sequence 5 nucleotide residues in length (5T's; estimated length of approximately 3.38 nm). Thymidines were used for the spacer regions (1) and (2). Each reporter region was 16 nucleotides in length.

Reporter probes were provided, individually comprising a red fluorescent dye and a reporter region complementary to the reporter hybridization region.

Adapter probes were designed similarly as illustrated in FIG. 1, comprising three amplifier probe hybridization regions separated by linkers (spacer regions). Three different spacer regions were used to test the effect of spacer regions of various lengths between the amplifier probe hybridization regions: (1) a sequence two nucleotide residues in length (2T's; estimated length of approximately 1.35 nm), (2) an 18-atom hexa-ethyleneglycol spacer (iSp18; estimated length of approximately 2.5 nm), and (3) a sequence 5 nucleotide residues in length (5T's; estimated length of approximately 3.38 nm). Thymidines were used for the spacer regions (1) and (2).

Primary probes were designed to hybridize to target sequences in Prox1 messenger RNA. For this experiment, ten primary probes were used to target Prox1 mRNA. Each primary probe comprised a first overhang and a second overhang, each overhang comprising an adapter hybridization region for hybridization of an adapter probe.

Mouse brain tissue sections were prepared by fixation followed by permeabilization and washed. Primary probes were contacted with the tissue sections and allowed to hybridize in hybridization buffer overnight at 37° C. Samples were incubated with blocking buffer, then adapter probes were hybridized in hybridization buffer at 34° C. for 30 minutes, followed by hybridization of amplifier probes in hybridization buffer at 34° C. for 30 minutes. Reporter probes were then hybridized at 30° C. for 30 minutes.

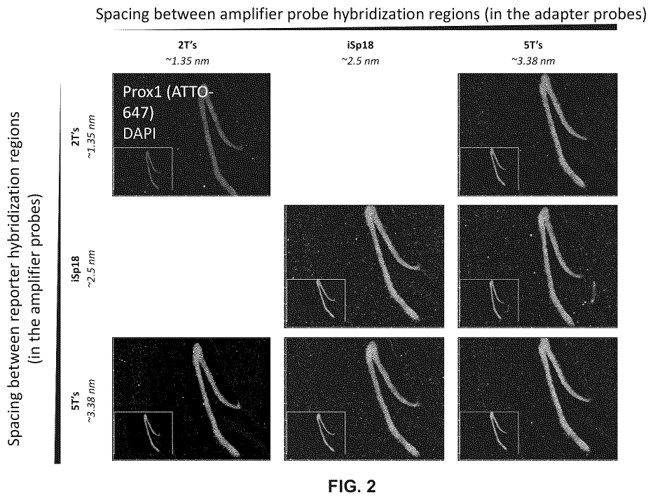

FIG. 2 provides fluorescent images for the detection of Prox1 mRNA in the mouse brain tissue sections using the various combinations of the different spacer regions between the reporter hybridization regions (spacers in the amplifier probes) and the different spacer regions between the amplifier probe hybridization regions (spacers in the adapter probes). The upper left panel of FIG. 2 shows the red dye signal detected for a complex comprising a 2T spacer between the amplifier probe hybridization regions and a 2T spacer between the reporter hybridization regions. Increasing the spacer length between the reporter probe hybridization regions to 5T without changing the spacer length in the adapter probes increased the signal intensity detected for Prox1, as shown in the bottom left panel of FIG. 2. Increasing the length of the spacer regions to 5T in both the amplifier probes and the adapter probes further increased the signal intensity (see bottom right panel of FIG. 2).

The organic spacer iSp18 also increased the red dye signal intensity compared to the 2T spacer. However, the iSp18 spacer generated more background noise compared to the nucleotide spacers.

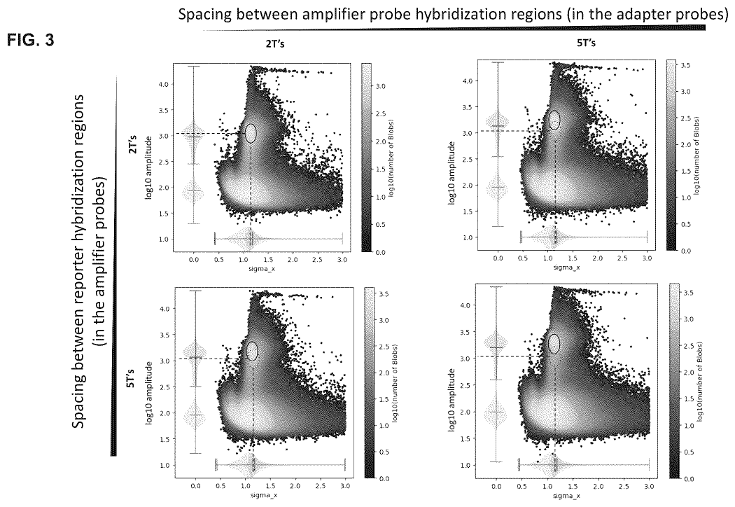

Amplitude sigma plots for the 2T and 5T spacers in the amplifier probes and adapter probes are provided in FIG. 3. The peak signal amplitude for the combination of 2T spacers in the amplifier probes and 2T spacers in the adapter probes is shown by the solid oval in the upper left panel of FIG. 3, and as a dashed oval in the other panels. As can be seen in the bottom panels of FIG. 3, compared to the top panels of FIG. 3, increasing the spacer length between the reporter hybridization regions (in the amplifier probes) from 2T to 5T increased the peak signal amplitude. Increasing the spacer length between the amplifier probe hybridization regions (in the adapter probes) from 2T to 5T also increased the peak signal amplitude.

These results demonstrate the benefits of a 5T spacer region compared to a 2T spacer region, for both spacer regions between reporter probe hybridization regions and spacer regions between adapter probe hybridization regions.

### Example 2: Increasing Spacer Length Up to 8 Nucleotides Improves Signal Intensity

This example provides results demonstrating increased signal intensity by using spacer lengths up to 8 nucleotides in length (8T) between multiple reporter hybridization regions in an amplifier probe, wherein the reporter hybridization regions hybridize to fluorescently labeled reporter probes. Signal intensity was further increased by using 8T spacers between multiple amplifier probe hybridization regions in an adapter probe.

Amplifier probes, reporter probes, adapter probes, and primary probes were designed as described in Example 1 above. To test whether further increasing the spacer length to 8 thymidine residues further improved signal intensity, amplifier probes and adapter probes were also designed with a spacer region of 8 thymidine residues (8T spacer; estimated length of approximately 5.4 nm). For this experiment, 50 primary probes were used to target Prox1 mRNA. Mouse brain tissue section preparation and probe hybridizations were performed as described in Example 1 above.

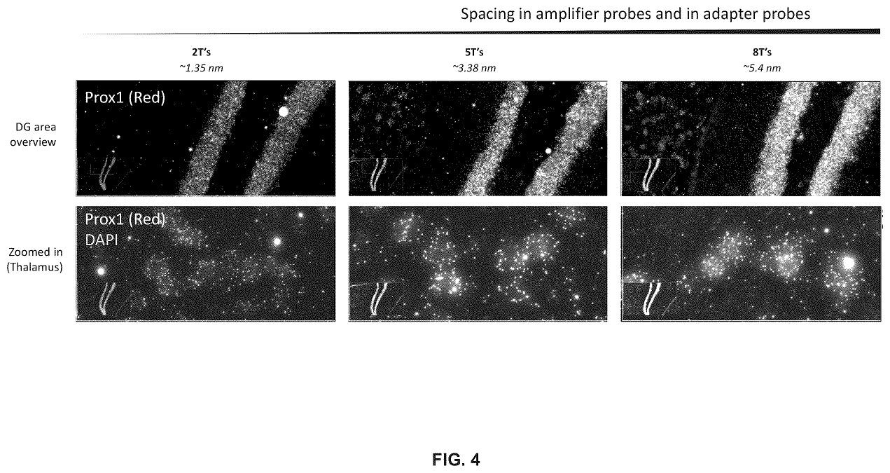

As shown in FIG. 4, a strong correlation between spacer length and signal intensity can be seen up to 8T spacer lengths (spacer sequence TTTTTTTT).

### Example 3: Increasing Spacer Length Improves Signal for Multiple Different Fluorophores

Examples 1 and 2 above demonstrated increased signal intensity using longer spacers between reporter probe hybridization regions and/or adapter probe hybridization regions for reporter probes labeled with a red fluorescent dye. This example provides results demonstrating increased signal intensity by using 5T spacers compared to 2T spacers for reporter probes comprising four different fluorescent dyes.

To test the effect of spacer length on signal intensity for different fluorescent dyes, reporter probes were prepared conjugated to four different fluorescent dyes (green, yellow, orange and red). Amplifier probes, reporter probes, adapter probes, and primary probes were designed as described in Example 1 above. For this experiment, 50 primary probes were used to target Prox1 mRNA. Mouse brain tissue section preparation and probe hybridizations were performed as described in Example 1 above.

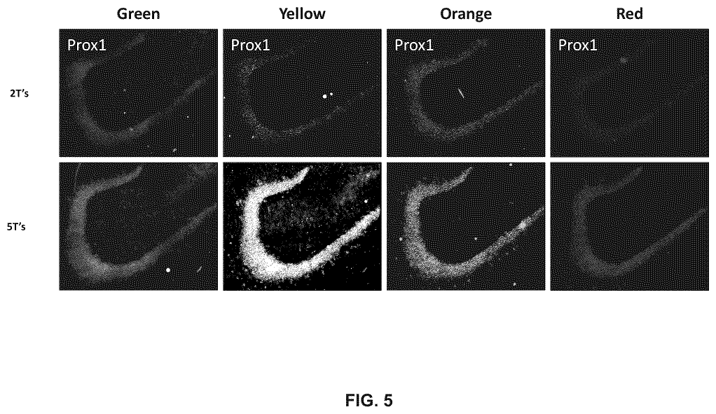

FIG. 5 provides fluorescent images for the detection of Prox1 mRNA in the mouse brain tissue sections using 2T or 5T spacers in the amplifier probes and the adapter probes for each of the dyes tested. As shown in FIG. 5, increasing the spacer length from 2T to 5T increased the signal intensity for all dyes tested. The increase in signal intensity was most dramatic for the yellow dye, followed by the orange dye, demonstrating that the increased spacer length is particularly helpful for certain dyes.

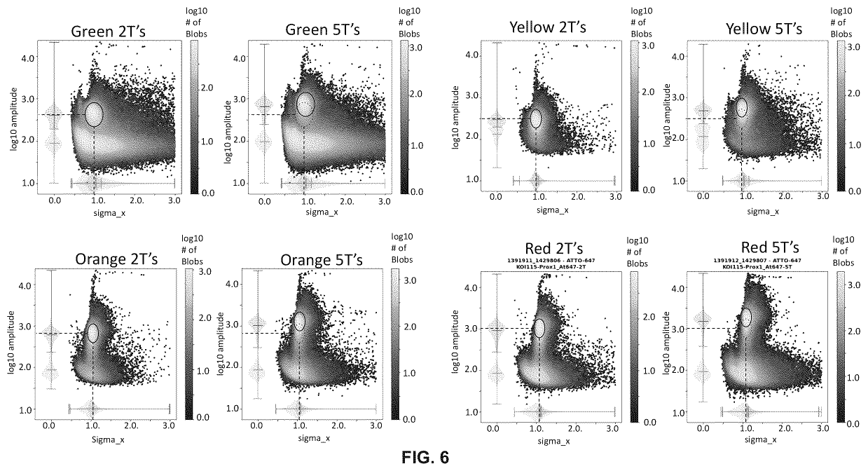

FIG. 6 provides amplitude-sigma plots showing how signal intensity in each color-channel benefits from increased spacer length. In the plots for the 5T spacers, the signal amplitude peak for the 2T spacer in the corresponding channel is shown as a dashed oval for comparison with the increased signal amplitude (solid oval).

### Example 4: Increasing Spacer Length in the Amplifier Probe Improves Signal Intensity

This example demonstrates the effect of variations in spacer length in primary (1°), adapter (L1), and amplifier (L2) probes on observed signal intensity from hybridized reporter probes labeled with a red dye. Prox1 mRNA was detected in mouse brain tissue sections as described in Example 1.

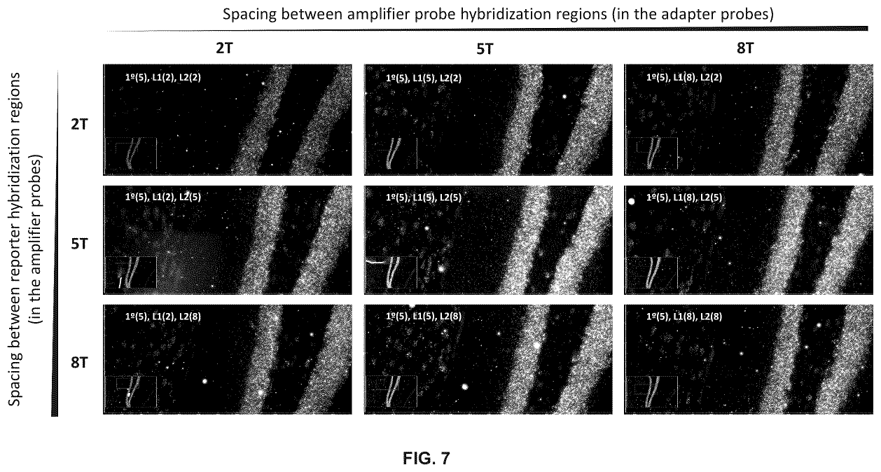

As shown in FIG. 7, spacer lengths of 2T, 5T, or 8T were incorporated into adapter and amplifier probes, designed as described in Example 1, and each variation was tested to determine which resulted in optimal signal intensity from the reporter probes. Improved signal intensities were detected as the spacer length in the amplifier probe was increased for each variation in adapter probe spacer length tested. Optimal results were obtained from a 5T spacer in the adapter probe and a 5T spacer in the amplifier probe.

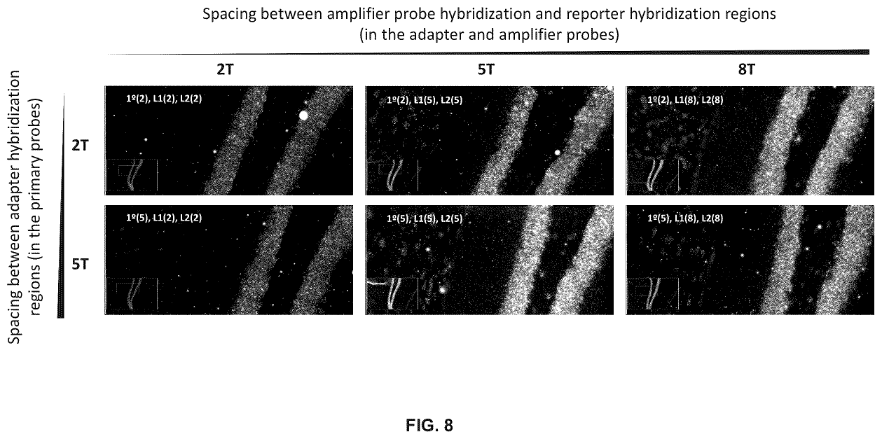

FIG. 8 provides fluorescent images for the detection of Prox1 mRNA in the mouse brain tissue sections using primary probes containing spacer sequences consisting of either 2T or 5T while varying the length of spacers in both adapter and amplifier probes to either 2T, 5T, or 8T. Optimal results were obtained when the primary, adapter, and amplifier probes contained 5T spacers. These results emphasize the importance of increased spacer length to reduce steric hinderance during probe assembly and dye-to-dye quenching during fluorescence imaging.

The present disclosure is not intended to be limited in scope to the particular disclosed embodiments, which are provided, for example, to illustrate various aspects of the present disclosure. Various modifications to the compositions and methods described will become apparent from the description and teachings herein. Such variations may be practiced without departing from the true scope and spirit of the disclosure and are intended to fall within the scope of the present disclosure.

1-84. (canceled)

85. A method for analyzing a biological sample, comprising:
(a) contacting the biological sample with:
(i) an amplifier probe comprising a recognition sequence and at least two reporter hybridization regions, wherein the at least two reporter hybridization regions are separated by a spacer region of between 4 and 8 nucleotides in length, and
(ii) a plurality of reporter probes, wherein each reporter probe of at least a subset of the reporter probes comprises a detectable label and a reporter region capable of hybridizing to the reporter hybridization region; and
(b) detecting a signal from a complex formed by hybridization of at least two reporter probes to the at least two reporter hybridization regions.

86. The method of claim 85, wherein the amplifier probe comprises, from 5′ to 3′ or from 3′ to 5′, the recognition sequence, a first reporter hybridization region, a first spacer region, a second reporter hybridization region, a second spacer region, and a third reporter hybridization region.

87. The method of claim 85, wherein the amplifier probe further comprises a linker sequence of between 2 and 8 nucleotides in length between the recognition sequence and the first reporter hybridization region.

88. The method of claim 85, wherein the spacer region is a sequence of thymidines.

89. The method of claim 86, wherein each spacer region is a sequence of thymidines and/or adenines, and wherein the spacer regions are the same.

90. The method of claim 85, wherein the reporter hybridization regions are individually between 15 and 25 nucleotides in length.

91. The method of claim 85, wherein the detectable label is a fluorophore.

92. The method of claim 91, wherein the fluorophore has an excitation peak between 520 nm and 540 nm.

93. The method of claim 91, wherein the fluorophore has an excitation peak between 590 nm and 600 nm.

94. The method of claim 91, wherein the detectable label is linked to the reporter region by one or more nucleotides.

95. The method of claim 85, wherein the recognition sequence is complementary to an amplifier probe hybridization region in an adapter probe, and wherein the method comprises contacting the biological sample with the adapter probe.

96. The method of claim 95, wherein the complex comprises between 2 and 20 amplifier probes hybridized to the adapter probe in the biological sample.

97. The method of claim 95, wherein the adapter probe comprises an adapter sequence capable of binding directly or indirectly to a primary probe hybridized to a target nucleic acid in the biological sample.

98. The method of claim 85, wherein the recognition sequence is complementary to an amplifier probe hybridization region in a primary probe hybridized to a target nucleic acid in the biological sample.

99. The method of claim 85, wherein the recognition sequence is complementary to a target sequence in a target nucleic acid in the biological sample.

100. The method of claim 99, wherein the target nucleic acid is an oligonucleotide reporter in a labeling agent bound to a non-nucleic acid analyte.

101. The method of claim 100, wherein the labeling agent is an antibody conjugated to the oligonucleotide reporter.

102. The method of claim 100, wherein the non-nucleic acid analyte is a protein.

103. The method of claim 100, wherein the target nucleic acid is a cellular nucleic acid analyte or a product thereof, or a probe or probe set associated with a nucleic acid analyte or product thereof in the biological sample.

104. The method of claim 85, wherein the biological sample is on a substrate.
